(19) United States
(12) Patent Application Publication
(10) Pub. No.: US 2018/0319717 A1
(43) Pub. Date: Nov. 8, 2018

(54) Processes and Systems for the Conversion of Acyclic Hydrocarbons

(71) Applicant: ExxonMobil Chemical Patents Inc., Baytown, TX (US)

(72) Inventors: Neeraj Sangar, League City, TX (US); Larry L. Iaccino, Seabrook, TX (US); Christopher L. Becker, Manhattan, KS (US)

(21) Appl. No.: 15/895,625

(22) Filed: Feb. 13, 2018

Related U.S. Application Data
(60) Provisional application No. 62/500,890, filed on May 3, 2017.

(51) Int. Cl. C07C 2/52; C07C 51/567; B01J 8/24

(52) U.S. Cl. CPC C07C 2/52; C07C 51/567; B01J 2208/00893; B01J 2208/00991; B01J 8/24

(57) ABSTRACT

This invention relates to processes and systems for converting acyclic hydrocarbons to alkenes, cyclic hydrocarbons and/or aromatics, for example converting acyclic C₅ hydrocarbons to cyclopentadiene in a reactor system. The process includes contacting a feedstock comprising acyclic hydrocarbons with a catalyst material and an inert material to convert at least a portion of the acyclic hydrocarbons to a first effluent comprising alkenes, cyclic hydrocarbons and/or aromatics. In particular, the catalyst material and the inert material have a different average diameter and/or density providing varying fluidization behavior in the reactor.

### CROSS-REFERENCE TO RELATED APPLICATION

This application claims priority to U.S. Provisional Application Ser. No. 62/500,890, filed May 3, 2017, the disclosure of which is incorporated herein by reference.

### FIELD OF THE INVENTION

This invention relates processes and reactor systems for the conversion of acyclic hydrocarbons to alkenes, cyclic hydrocarbons and/or aromatics.

### BACKGROUND OF THE INVENTION

Cyclic hydrocarbons, alkenes, and aromatics, such as cyclopentadiene (“CPD”) and its dimer dicyclopentadiene (“DCPD”), ethylene, propylene, and benzene, are highly desired raw materials used throughout the chemical industry in a wide range of products, for example, polymeric materials, polyester resins, synthetic rubbers, solvents, fuels, fuel additives, etc. These compounds are typically derived from various streams produced during refinery processing of petroleum. In particular, CPD is currently a minor byproduct of liquid fed steam cracking (e.g., naphtha and heavier feed). As existing and new steam cracking facilities shift to lighter feeds, less CPD is produced while demand for CPD is rising. High cost due to supply limitations impacts the potential end product use of CPD in polymers. More CPD-based polymer product could be produced if additional CPD could be produced at unconstrained rates and preferably at a cost lower than recovery from steam cracking. When producing CPD, co-production of other cyclic C₅ compounds is also desirable. In particular, cyclopentane and cyclopentene can have high value as solvents while cyclopentene may be used as a comonomer to produce polymers and as a starting material for other high value chemicals.

It would be advantageous to be able to produce these cyclic hydrocarbons, alkenes, and aromatics, including CPD, propylene, ethylene, and benzene, as the primary product from plentiful hydrocarbon feedstock. When producing CPD, it is also desirable to minimize production of light (C₄₋) byproducts. While a feedstock composed of lower hydrogen content (e.g., cyclics, alkenes, and dialkenes) could be preferred because the reaction endotherm is reduced and thermodynamic constraints on conversion are improved, non-saturates are more expensive than saturate feedstock. Further, an abundance of saturate hydrocarbons, such as C₅ hydrocarbons, are available from unconventional gas and shale oil, as well as reduced use in motor fuels due to stringent environmental regulations. Various hydrocarbon feedstocks, such as C₅ feedstock, may also be derived from bio-feeds. Linear hydrocarbon skeletal structure is preferred over branched hydrocarbon skeletal structures due to both reaction chemistry and the lower value of linear hydrocarbon relative to branched hydrocarbon (due to octane differences).

Various catalytic dehydrogenation technologies are currently used to produce mono- and di-olefins from C₃ and C₄ alkanes, but not cyclic mono-olefins or cyclic di-olefins. A typical process uses Pt/Sn supported on alumina as the active catalyst. Another useful process uses chromia on alumina. See, B. V. Vora, “Development of Dehydrogenation Catalysts and Processes,” Topics in Catalysis, vol. 55, pp. 1297-1308, 2012; and J. C. Bricker, “Advanced Catalytic Dehydrogenation Technologies for Production of Olefins,” Topics in Catalysis, vol. 55, pp. 1309-1314, 2012.

Still another common process uses Pt/Sn supported on Zn and/or Ca aluminate to dehydrogenate propane. While these processes are successful in dehydrogenating alkanes, they do not perform cyclization, which is critical to producing CPD. Pt—Sn/alumina and Pt—Sn/aluminate catalysts exhibit moderate conversion of n-pentane, but such catalyst have poor selectivity and yield to cyclic C₅ products.

Pt supported on chlorided alumina catalysts are used to reform low octane naphtha to aromatics such as benzene and toluene. See, U.S. Pat. No. 3,953,368 (Sinfelt), “Polymetallic Cluster Compositions Useful as Hydrocarbon Conversion Catalysts.” While these catalysts are effective in dehydrogenating and cyclizing C₆ and higher alkanes to form C₆ aromatic rings, they are less effective in converting acyclic C₅s to cyclic C₅s. These Pt supported on chlorided alumina catalysts exhibit low yields of cyclic C₅ and exhibit deactivation within the first two hours of time on stream. Cyclization of C₆ and C₇ alkanes is aided by the formation of an aromatic ring, which does not occur in C₅ cyclization. This effect may be due in part to the much higher heat of formation for CPD, a cyclic C₅, as compared to benzene, a cyclic C₆, and toluene, a cyclic C₇. This is also exhibited by Pt/Ir and Pt/Sn supported on chlorided alumina. Although these alumina catalysts perform both dehydrogenation and cyclization of C₆₊ species to form C₆ aromatic rings, a different catalyst will be needed to convert acyclic C₅ to cyclic C₅.

Ga-containing ZSM-5 catalysts are used in a process to produce aromatics from light paraffins. A study by Kanazirev et al. showed n-pentane is readily converted over Ga₂O₃/H-ZSM-5. See Kanazirev Price et al., “Conversion of C8 aromatics and n-pentane over Ga₂O₃/H-ZSM-5 mechanically mixed catalysts,” Catalysis Letters, vol. 9, pp. 35-42, 1991. No production of cyclic C₅ was reported while upwards of 6 wt % aromatics were produced at 440° C. and 1.8 hr⁻¹ WHSV. Mo/ZSM-5 catalysts have also been shown to dehydrogenate and/or cyclize paraffins, especially methane. See, Y. Xu, S. Liu, X. Guo, L. Wang, and M. Xie, “Methane activation without using oxidants over Mo/HZSM-5 zeolite catalysts,” Catalysis Letters, vol. 30, pp. 135-149, 1994. High conversion of n-pentane using Mo/ZSM-5 was demonstrated with no production of cyclic C₅ and high yield to cracking products. This shows that ZSM-5-based catalysts can convert paraffins to a C₆ ring, but not necessarily to produce a C₅ ring.

U.S. Pat. No. 5,254,787 (Dessau) introduced the NU-87 catalyst used in the dehydrogenation of paraffins. This catalyst was shown to dehydrogenate C₂-C₆₊ to produce their unsaturated analogs. A distinction between C₂₋₅ and C₆₊ alkanes was made explicit in this patent: dehydrogenation of C₂₋₅ alkanes produced linear or branched mono-olefins or di-olefins, whereas dehydrogenation of C₆₊ alkanes yielded aromatics. U.S. Pat. No. 5,192,728 (Dessau) involves similar chemistry, but with a tin-containing crystalline microporous material. As with the NU-87 catalyst, C₅ dehydrogenation was only shown to produce linear or branched, mono-olefins or di-olefins and not CPD.

U.S. Pat. No. 5,284,986 (Dessau) introduced a dual-stage process for the production of cyclopentane and cyclopentene from n-pentane. An example was conducted wherein the first stage involved dehydrogenation and dehydrocyclization of n-pentane to a mix of paraffins, mono-olefins and di-olefins, and naphthenes over a Pt/Sn-ZSM-5 catalyst. This mixture was then introduced to a second-stage reactor consisting of Pd/Sn-ZSM-5 catalyst where dienes, especially CPD, were converted to olefins and saturates. Cyclopentene was the desired product in this process, whereas CPD was an unwanted byproduct.

U.S. Pat. Nos. 2,438,398; 2,438,399; 2,438,400; 2,438,401; 2,438,402; 2,438,403; and U.S. Pat. No. 2,438,404 (Kennedy) disclosed production of CPD from 1,3-pentadiene over various catalysts. Low operating pressures, low per pass conversion, and low selectivity make this process undesirable. Additionally, 1,3-pentadiene is not a readily available feedstock, unlike n-pentane. See also, Kennedy et al., “Formation of Cyclopentadiene from 1,3-Pentadiene,” Industrial & Engineering Chemistry, vol. 42, pp. 547-552, 1950.

Fel'dblyum et al. in “Cyclization and dehydrocyclization of C₅ hydrocarbons over platinum nanocatalysts and in the presence of hydrogen sulfide,” Doklady Chemistry, vol. 424, pp. 27-30, 2009, reported production of CPD from 1,3-pentadiene, n-pentene, and an n-pentane. Yields to CPD were as high as 53%, 35%, and 21% for the conversion of 1,3-pentadiene, n-pentene, and an n-pentane, respectively, at 600° C. on 2% Pt/SiO₂. While initial production of CPD was observed, drastic catalyst deactivation within the first minutes of the reaction was observed. Experiments conducted on Pt-containing silica show moderate conversion of n-pentane over Pt—Sn/SiO₂, but with poor selectivity and yield to cyclic C₅ products. The use of H₂S as a 1,3-pentadiene cyclization promoter was presented by Fel'dblyum, infra, as well as in Marcinkowski, “Isomerization and Dehydrogenation of 1,3-Pentadiene,” M.S., University of Central Florida, 1977. Marcinkowski showed 80% conversion of 1,3,-pentadiene with 80% selectivity to CPD with H₂S at 700° C. High temperature, limited feedstock, and potential of products containing sulfur that would later need scrubbing make this process undesirable.

Lopez et al. in “n-Pentane Hydroisomerization on Pt Containing HZSM-5, HBEA, and SAPO-11,” Catalysis Letters, vol. 122, pp. 267-273, 2008, studied reactions of n-pentane on Pt-containing zeolites including H-ZSM-5. At intermediate temperatures (250° C.-400° C.), they reported efficient hydroisomerization of n-pentane on the Pt-zeolites with no discussion of cyclopentene formation. It is desirable to avoid this deleterious chemistry as branched C₅ do not produce cyclic C₅ as efficiently as linear C₅, as discussed above.

Li et al. in “Catalytic dehydroisomerization of n-alkanes to isoalkenes,” Journal of Catalysis, vol. 255, pp. 134-137, 2008, also studied n-pentane dehydrogenation on Pt-containing zeolites in which Al had been isomorphically substituted with Fe. These Pt/[Fe]ZSM-5 catalysts were efficient dehydrogenating and isomerizing n-pentane, but under the reaction conditions used, no cyclic C₅ were produced and undesirable skeletal isomerization occurred.

U.S. Pat. No. 5,633,421 discloses a process for dehydrogenating C₂-C₅ paraffins to obtain corresponding olefins. Similarly, U.S. Pat. No. 2,982,798 discloses a process for dehydrogenating an aliphatic hydrocarbon containing 3 to 6, inclusive, carbon atoms. However, neither U.S. Pat. No. 5,633,421 nor U.S. Pat. No. 2,982,798 disclose production of CPD from acyclic C₅ hydrocarbons, which are desirable as feedstock because they are plentiful and low cost.

Further, on-purpose production of CPD, propylene, ethylene, and benzene is accomplished via endothermic reactions. Engineering process and reactor design for catalyst driven endothermic reactions presents many challenges. For example, maintaining high temperatures required for the reactions including transferring a large amount of heat to a catalyst can be difficult. Production of CPD is especially difficult amongst endothermic processes because it is favored by low pressure and high temperature, but competing reactions such as cracking of n-pentane and other C₅ hydrocarbons can occur at relatively low temperature (e.g., 450° C.-500° C.).

Additional challenges may include loss of catalyst activity due to coking during the process and further processing needed to remove coke from the catalyst, and the inability to use oxygen-containing gas to directly provide the heat input necessary to counter the endothermic nature of the reaction without damaging the catalyst. Moreover, non-uniform catalyst aging can also occur, which can impact resulting product selectivity and catalyst life.

Furthermore, challenges exist in reactor design, especially with respect to material selection, since the reactions are carried out at higher temperatures and highly carburizing conditions. Metal alloys can potentially undergo carburization (resulting in loss in mechanical properties) as well as metal dusting (resulting in loss of metal via formation of metastable carbides) under the desired reaction conditions. Thus, given the need for large heat input to drive the reaction, presence of metallic heat-transfer surfaces exposed to the reaction mixture need to be capable of resisting attack via carburization/metal dusting.

Hence, there remains a need for a process to convert acyclic hydrocarbons to alkenes, cyclic hydrocarbons, and aromatics, particularly acyclic C₅ hydrocarbon to CPD, preferably at commercial rates and conditions. Further, there is a need for a catalytic process targeted for the production of CPD, which generates CPD in high yield from plentiful C₅ feedstocks without excessive production of C₄₋ cracked products and with acceptable catalyst aging properties. Additionally, there is a need for processes and systems for on-purpose production of CPD, propylene, ethylene, and benzene from acyclic hydrocarbons, which addresses the above-described challenges.

### SUMMARY OF THE INVENTION

In one aspect, this invention relates to a process for converting acyclic hydrocarbons to alkenes, cyclic hydrocarbons and/or aromatics in a reactor system, wherein the process comprises: contacting a feedstock comprising acyclic hydrocarbons with a catalyst material and an inert material in at least one reaction zone under reaction conditions to convert at least a portion of the acyclic hydrocarbons to a first effluent comprising alkenes, cyclic hydrocarbons, and/or aromatics, wherein the catalyst material and the inert material have a different average diameter and/or density; removing an inert material-rich stream from the at least one reaction zone; optionally, separating catalyst material from the inert material-rich stream; heating the inert material-rich stream to produce a heated inert material-rich stream; and providing the heated inert material-rich stream to the at least one reaction zone.

In another aspect, this invention also relates to a reaction system for converting acyclic hydrocarbons to alkenes, cyclic hydrocarbons and/or aromatics, wherein the reaction system comprises: a feedstock stream comprising acyclic hydrocarbons; an effluent stream comprising alkenes, cyclic hydrocarbons and/or aromatics; an inert material-rich stream; a heated inert material-rich stream; a separated catalyst material stream; and a catalyst-free effluent stream; at least one reactor operated under reaction conditions to convert at least a portion of the acyclic hydrocarbons to alkenes, cyclic hydrocarbons and/or aromatics, wherein the at least one reactor comprises: a feedstock stream inlet; a heated inert material-rich stream inlet; a separated catalyst material stream inlet; an effluent stream outlet; and an inert material-rich stream outlet; a first separator for separating catalyst material from the effluent stream to produce the separated catalyst material stream and the catalyst-free effluent stream, wherein the first separator is in fluid connection with the at least one reactor and comprises an effluent stream inlet, a separated catalyst material stream outlet and a catalyst-free effluent stream outlet; and a second separator for separating catalyst material from the inert material-rich stream, wherein the second separator is in fluid connection with the at least one reactor.

### BRIEF DESCRIPTION OF THE FIGURES

FIG. 1 is a diagram of a reactor system according to an embodiment of the invention.

FIG. 2 is a diagram of a reactor system according to another embodiment of the invention.

Corresponding reference numerals indicate corresponding parts throughout the several views of the drawings.

### DETAILED DESCRIPTION OF THE INVENTION

All numerical values within the detailed description and the claims herein are modified by “about” or “approximately” the indicated value, and take into account experimental error and variations that would be expected by a person having ordinary skill in the art. Unless otherwise indicated, room temperature is about 23° C.

### I. DEFINITIONS

To facilitate an understanding of the present invention, a number of terms and phrases are defined below.

As used in the present disclosure and claims, the singular forms “a,” “an,” and “the” include plural forms unless the context clearly dictates otherwise.

The term “and/or” as used in a phrase such as “A and/or B” herein is intended to include “A and B,” “A or B,” “A”, and “B.”

As used herein, the term “about” refers to a range of values of plus or minus 10% of a specified value. For example, the phrase “about 200” includes plus or minus 10% of 200, or from 180 to 220.

The term “hydrocarbon” means a class of compounds containing hydrogen bound to carbon, and encompasses (i) saturated hydrocarbon compounds, (ii) unsaturated hydrocarbon compounds, and (iii) mixtures of hydrocarbon compounds (saturated and/or unsaturated), including mixtures of hydrocarbon compounds having different values of n. The term “Cₙ” means hydrocarbon(s) having n carbon atom(s) per molecule, wherein n is a positive integer.

As used herein, the term “light hydrocarbon” means light paraffinic and/or olefinic hydrocarbons comprised substantially of hydrogen and carbon only and has one to no more than 4 carbon atoms.

The term “saturates” includes, but is not limited to, alkanes and cycloalkanes.

The term “non-saturates” includes, but is not limited to, alkenes, dialkenes, alkynes, cyclo-alkenes, and cyclo-dialkenes.

The term “cyclic hydrocarbon” denotes groups such as the cyclopropane, cyclopropene, cyclobutane, cyclobutadiene, etc., and substituted analogues of these structures. These cyclic hydrocarbons can be single- or multi-ring structures. Preferably, the term “cyclic hydrocarbon” refers to non-aromatics.

The term “cyclics C₅” or “cC₅” includes, but is not limited to, cyclopentane, cyclopentene, cyclopentadiene, and mixtures of two or more thereof. The term “cyclic C₅” or “cC₅” also includes alkylated analogs of any of the foregoing, e.g., methyl cyclopentane, methyl cyclopentene, and methyl cyclopentadiene. It should be recognized for purposes of the invention that cyclopentadiene spontaneously dimerizes over time to form dicyclopentadiene via Diels-Alder condensation over a range of conditions, including ambient temperature and pressure.

The term “acyclics” includes, but is not limited to, linear and branched saturates and non-saturates.

The term “alkane” refers to non-aromatic saturated hydrocarbons with the general formula CₙH₍₂ₙ₊₂₎, where n is 1 or greater. An alkane may be straight chained or branched. Examples of alkanes include, but are not limited to methane, ethane, propane, butane, pentane, hexane, heptane and octane. “Alkane” is intended to embrace all structural isomeric forms of an alkane. For example, butane encompasses n-butane and isobutane; pentane encompasses n-pentane, isopentane and neopentane.

The term “alkene,” alternatively referred to as “olefin,” refers to a branched or unbranched unsaturated hydrocarbon having one or more carbon-carbon double bonds. A simple alkene comprises the general formula CₙH₂ₙ, where n is 2 or greater. Examples of alkenes include, but are not limited to ethylene, propylene, butylene, pentene, hexene and heptene. “Alkene” is intended to embrace all structural isomeric forms of an alkene. For example, butylene encompasses but-1-ene, (Z)-but-2-ene, etc.

The term “aromatic” means a planar cyclic hydrocarbyl with conjugated double bonds, such as benzene. As used herein, the term aromatic encompasses compounds containing one or more aromatic rings, including, but not limited to, benzene, toluene, and xylene, and polynuclear aromatics (PNAs), which include naphthalene, anthracene, chrysene, and their alkylated versions. The term “C₆₊ aromatics” includes compounds based upon an aromatic ring having six or more ring atoms, including, but not limited to, benzene, toluene, and xylene, and polynuclear aromatics (PNAs), which include naphthalene, anthracene, chrysene, and their alkylated versions.

The term “BTX” includes, but is not limited to, a mixture of benzene, toluene, and xylene (ortho and/or meta and/or para).

The term “coke” includes, but is not limited to, a low hydrogen content hydrocarbon that is adsorbed on the catalyst composition.

The term “Cₙ₊” means hydrocarbon(s) having at least n carbon atom(s) per molecule.

The term “Cₙ₋” means hydrocarbon(s) having no more than n carbon atom(s) per molecule.

The term “C₅ feedstock” includes a feedstock containing n-pentane, such as a feedstock which is predominately normal pentane and isopentane (also referred to as methylbutane), with smaller fractions of cyclopentane and neopentane (also referred to as 2,2-dimethylpropane).

All numbers and references to the Periodic Table of Elements are based on the new notation as set out in Chemical and Engineering News, 63(5), 27 (1985), unless otherwise specified.

The term “Group 10 metal” means an element in Group 10 of the Periodic Table and includes, but is not limited to, Ni, Pd, and Pt, and a mixture of two or more thereof.

The term “Group 11 metal” means an element in Group 11 of the Periodic Table and includes, but is not limited to, Cu, Ag, Au, and a mixture of two or more thereof.

The term “Group 1 alkali metal” means an element in Group 1 of the Periodic Table and includes, but is not limited to, Li, Na, K, Rb, C₅, and a mixture of two or more thereof, and excludes hydrogen.

The term “Group 2 alkaline earth metal” means an element in Group 2 of the Periodic Table and includes, but is not limited to, Be, Mg, Ca, Sr, Ba, and a mixture of two or more thereof.

The term “rare earth metal” means an element in the Lanthanide series of the Periodic Table, as well as scandium and yttrium. The term rare earth metal includes, but is not limited to, lanthanum, praseodymium, neodymium, cerium, yttrium, and a mixture of two or more thereof.

The term “oxygen” includes air, O₂, H₂O, CO, and CO₂.

The term “constraint index” is defined in U.S. Pat. No. 3,972,832 and U.S. Pat. No. 4,016,218, both of which are incorporated herein by reference.

As used herein, the term “molecular sieve of the MCM-22 family” (or “material of the MCM-22 family” or “MCM-22 family material” or “MCM-22 family zeolite”) includes one or more of:
molecular sieves made from a common first degree crystalline building block unit cell, which unit cell has the MWW framework topology. (A unit cell is a spatial arrangement of atoms, which if tiled in three-dimensional space describes the crystal structure. Such crystal structures are discussed in the “Atlas of Zeolite Framework Types”, Fifth edition, 2001, the entire content of which is incorporated as reference);
molecular sieves made from a common second degree building block, being a 2-dimensional tiling of such MWW framework topology unit cells, forming a monolayer of one unit cell thickness, preferably one c-unit cell thickness;
molecular sieves made from common second degree building blocks, being layers of one or more than one unit cell thickness, wherein the layer of more than one unit cell thickness is made from stacking, packing, or binding of at least two monolayers of one unit cell thickness. The stacking of such second degree building blocks may be in a regular fashion, an irregular fashion, a random fashion, or any combination thereof; and
molecular sieves made by any regular or random 2-dimensional or 3-dimensional combination of unit cells having the MWW framework topology.

The MCM-22 family includes those molecular sieves having an X-ray diffraction pattern including d-spacing maxima at 12.4±0.25, 6.9±0.15, 3.57±0.07, and 3.42±0.07 Angstrom. The X-ray diffraction data used to characterize the material are obtained by standard techniques using the K-alpha doublet of copper as incident radiation and a diffractometer equipped with a scintillation counter and associated computer as the collection system.

As used herein, the term “molecular sieve” is used synonymously with the term “microporous crystalline material” or “zeolite.”

As used herein, the term “selectivity” means the moles of carbon in the respective cyclic C₅, CPD, C₁, and C₂-4 formed divided by total moles of carbon in the pentane converted. For example, the term “carbon selectivity to cyclic C₅ of at least 30%” means that at least 30 moles of carbon in the cyclic C₅ is formed per 100 moles of carbon in the pentane converted.

As used herein, the term “conversion” means the moles of carbon in the acyclic C₅ feedstock that is converted to a product. The phrase “a conversion of at least 70% of said acyclic C₅ feedstock to said product” means that at least 70% of the moles of said acyclic C₅ feedstock was converted to a product.

As used herein, the term “Alpha Value” is used as a measure of the cracking activity of a catalyst and is described in U.S. Pat. No. 3,354,078 and in the Journal of Catalysis, Vol. 4, p. 527 (1965); Vol. 6, p. 278, (1966) and Vol. 61, p. 395, (1980), each incorporated herein by reference. The experimental conditions of the test used herein included a constant temperature of 538° C. and a variable flow rate as described in detail in the Journal of Catalysis, Vol. 61, p. 395, (1980).

As used herein, the term “reactor system” refers to a system including one or more reactors and all necessary and optional equipment used in the production of cyclopentadiene.

As used herein, the term “reactor” refers to any vessel(s) in which a chemical reaction occurs. Reactor includes both distinct reactors, as well as reaction zones within a single reactor apparatus and, as applicable, reactions zones across multiple reactors. In other words, and as is common, a single reactor may have multiple reaction zones. Where the description refers to a first and second reactor, the person of ordinary skill in the art will readily recognize such reference includes two reactors, as well as a single reactor vessel having first and second reaction zones. Likewise, a first reactor effluent and a second reactor effluent will be recognized to include the effluent from the first reaction zone and the second reaction zone of a single reactor, respectively.

A reactor/reaction zone may be an adiabatic reactor/reaction zone or a diabatic reactor/reaction zone. As used herein, the term “adiabatic” refers to a reaction zone for which there is essentially no heat input into the system other than by a flowing process fluid. A reaction zone that has unavoidable losses due to conduction and/or radiation may also be considered adiabatic for the purpose of this invention. As used herein, the term “diabatic” refers to a reactor/reaction zone to which heat is supplied by a means in addition to that provided by the flowing process fluid.

As used herein, the term “moving bed” reactor refers to a zone or vessel with contacting of solids (e.g., catalyst particles) and gas flows such that the superficial gas velocity (U) is below the velocity required for dilute-phase pneumatic conveying of solid particles in order to maintain a solids bed with void fraction below 95%. In a moving bed reactor, the solids (e.g., catalyst material) may slowly travel through the reactor and may be removed from the bottom of the reactor and added to the top of the reactor. A moving bed reactor may operate under several flow regimes including settling or moving packed-bed regime (U<U_{mf}), bubbling regime (U_{mf}<U<U_{mb}), slugging regime (U_{mb}<U<U_{c}), transition to and turbulent fluidization regime (U_{c}<U<Uₜᵣ), and fast-fluidization regime (U>Uₜᵣ), where U_{mf} is minimum fluidizing velocity, U_{mb} is minimum bubbling velocity, Uc is the velocity at which fluctuation in pressure peaks, and tr is transport velocity. These different fluidization regimes have been described in, for example, Kunii, D., Levenspiel, O., Chapter 3 of Fluidization Engineering, 2^{nd} Edition, Butterworth-Heinemann, Boston, 1991 and Walas, S. M., Chapter 6 of Chemical Process Equipment, Revised 2^{nd} Edition, Butterworth-Heinemann, Boston, 2010, which are incorporated by reference.

As used herein, the term “settling bed” reactor refers to a zone or vessel wherein particulates contact with gas flows such that the superficial gas velocity (U) is below the minimum velocity required to fluidize the solid particles (e.g., catalyst particles), the minimum fluidization velocity (U_{mf}), U<U_{mf}, in at least a portion of the reaction zone, and/or operating at a velocity higher than the minimum fluidization velocity while maintaining a gradient in gas and/or solid property (such as, temperature, gas, or solid composition, etc.) axially up the reactor bed by using reactor internals to minimize gas-solid back-mixing. Description of the minimum fluidization velocity is given in, for example, Kunii, D., Levenspiel, O., Chapter 3 of Fluidization Engineering, 2^{nd} Edition, Butterworth-Heinemann, Boston, 1991 and Walas, S. M., Chapter 6 of Chemical Process Equipment, Revised 2^{nd} Edition, Butterworth-Heinemann, Boston, 2010. A settling bed reactor may be a “circulating settling bed reactor,” which refers to a settling bed with a movement of solids (e.g., catalyst material) through the reactor and at least a partial recirculation of the solids (e.g., catalyst material). For example, the solids (e.g., catalyst material) may have been removed from the reactor, regenerated, reheated, and/or separated from the product stream and then returned back to the reactor.

As used herein, the term “fluidized bed” reactor refers to a zone or vessel with contacting of solids (e.g., catalyst particles) and gas flows such that the superficial gas velocity (U) is sufficient to fluidize solid particles (i.e., above the minimum fluidization velocity U_{mf}) and is below the velocity required for dilute-phase pneumatic conveying of solid particles in order to maintain a solids bed with void fraction below 95%. As used herein, the term “cascaded fluid-beds” means a series arrangement of individual fluid-beds such that there can be a gradient in gas and/or solid property (such as, temperature, gas, or solid composition, pressure, etc.) as the solid or gas cascades from one fluid-bed to another. Locus of minimum fluidization velocity is given in, for example, Kunii, D., Levenspiel, O., Chapter 3 of Fluidization Engineering, 2^{nd} Edition, Butterworth-Heinemann, Boston, 1991 and Walas, S. M., Chapter 6 of Chemical Process Equipment, Revised 2^{nd} Edition, Butterworth-Heinemann, Boston, 2010. A fluidized bed reactor may be a moving fluidized bed reactor, such as a “circulating fluidized bed reactor,” which refers to a fluidized bed with a movement of solids (e.g., catalyst material) through the reactor and at least a partial recirculation of the solids (e.g., catalyst material). For example, the solids (e.g., catalyst material) may have been removed from the reactor, regenerated, reheated, and/or separated from the product stream and then returned back to the reactor.

As used herein, the term “riser” reactor (also known as a transport reactor) refers to a zone or vessel (such as, vertical cylindrical pipe) used for net upwards transport of solids (e.g., catalyst particles) in fast-fluidization or pneumatic conveying fluidization regimes. Fast fluidization and pneumatic conveying fluidization regimes are characterized by superficial gas velocities (U) greater than the transport velocity (Uₜᵣ). Fast fluidization and pneumatic conveying fluidization regimes are also described in Kunii, D., Levenspiel, O., Chapter 3 of Fluidization Engineering, 2^{nd} Edition, Butterworth-Heinemann, Boston, 1991 and Walas, S. M., Chapter 6 of Chemical Process Equipment, Revised 2^{nd} Edition, Butterworth-Heinemann, Boston, 2010. A fluidized bed reactor, such as a circulating fluidized bed reactor, may be operated as a riser reactor.

“Average diameter” for particles in the range of 1 to 3500 μm is determined using a Mastersizer™ 3000 available from Malvern Instruments, Ltd., Worcestershire, England. Unless otherwise stated, particle size is determined at D50. D50 is the value of the particle diameter at 50% in the cumulative distribution. For example, if D50=5.8 um, then 50% of the particles in the sample are equal to or larger than 5.8 um and 50% are smaller than 5.8 um. (In contrast, if D90=5.8 um, then 10% of the particles in the sample are larger than 5.8 um and 90% are smaller than 5.8 um.) “Average diameter” for particles in the range of 3 mm to 50 mm is determined using a micrometer on a representative sample of 100 particles.

“Particle density” of a particulate material refers to the density of the constituent particles, not including the void space between the particles. For the avoidance of doubt, particle density does account for any voids or porosity contained within the particles.

For purposes of the invention, 1 psi is equivalent to 6.895 kPa. Particularly, 1 psia is equivalent to 1 kPa absolute (kPa-a). Likewise, 1 psig is equivalent to 6.895 kPa gauge (kPa-g).

### II. ACYCLIC HYDROCARBON CONVERSION PROCESS

In a first aspect, this invention relates to a process for converting acyclic hydrocarbons to alkenes, cyclic hydrocarbons and/or aromatics in a reactor system. The process may comprise contacting a feedstock comprising acyclic hydrocarbons with a catalyst material and an inert material in at least one reaction zone under reaction conditions to convert at least a portion of the acyclic hydrocarbons to a first effluent comprising alkenes, cyclic hydrocarbons and/or aromatics, removing an inert material-rich stream from the at least one reaction zone, separating catalyst material from the inert material-rich stream, heating the inert material-rich stream to produce a heated inert material-rich stream, and providing the heated inert material-rich stream to the at least one reaction zone. In various aspects, the catalyst material and the inert material preferably may have a different average diameter and/or density.

In one or more embodiments, this invention relates to a process for conversion of an acyclic C₅ feedstock to a product comprising cyclic C₅ compounds (e.g., cyclopentadiene). The process comprising the steps of contacting said feedstock and, optionally, hydrogen under acyclic C₅ conversion conditions in the presence of one or more catalyst compositions and inert materials, including but not limited to the catalyst compositions described herein and inert materials described herein, to form said product.

In one or more embodiments, the product of the process for conversion of an acyclic C₅ feedstock comprises cyclic C₅ compounds. The cyclic C₅ compounds comprise one or more of cyclopentane, cyclopentene, cyclopentadiene, and includes mixtures thereof. In one or more embodiments, the cyclic C₅ compounds comprise at least about 20 wt %, or 30 wt %, or 40 wt %, or 70 wt % cyclopentadiene, or in the range of from about 10 wt % to about 80 wt %, alternately 20 wt % to 70 wt %.

In one or more embodiments, the acyclic C₅ conversion conditions include at least a temperature, an n-pentane partial pressure, and a weight hourly space velocity (WHSV). The temperature is in the range of about 400° C. to about 700° C., or in the range from about 450° C. to about 650° C., preferably, in the range from about 500° C. to about 600° C. The n-pentane partial pressure is in the range of about 3 to about 100 psia at the reactor inlet, or in the range from about 3 to about 50 psia, preferably, in the range from about 3 psia to about 20 psia. The weight hourly space velocity is in the range from about 1 to about 50 hr⁻¹, or in the range from about 1 to about 20 hr⁻¹. Such conditions include a molar ratio of the optional hydrogen co-feed to the acyclic C₅ feedstock in the range of about 0 to 3, or in the range from about 1 to about 2. Such conditions may also include co-feed C₁-C₄ hydrocarbons with the acyclic C₅ feed.

In one or more embodiments, this invention relates to a process for conversion of n-pentane to cyclopentadiene comprising the steps of contacting n-pentane and, optionally, hydrogen (if present, typically H₂ is present at a ratio to n-pentane of 0.01 to 3.0) with one or more catalyst compositions and inert materials, including but not limited to the catalyst compositions described herein and the inert materials described herein, to form cyclopentadiene at a temperature of 400° C. to 700° C., an n-pentane partial pressure of 3 to about 100 psia at the reactor inlet, and a weight hourly space velocity of 1 to about 50 hr⁻¹.

### A. Feedstock

In the process, a feedstock comprising acyclic hydrocarbons, preferably acyclic C₂-C₁₀ hydrocarbons are provided to a reactor system comprising a catalyst material and an inert material. Acyclic C₂-C₁₀ hydrocarbons include, but are not limited to alkanes (e.g., ethane, propane, butane, pentane, hexane, etc.), alkenes (e.g., ethylene, propylene, butylene, etc.), alkynes (e.g., ethyne, propyne, 1-butyne, 2-butyne, etc.), dialkenes (e.g., 1,2-propadiene, 1,3-butadiene, 1,3-pentadiene, etc.) and combinations thereof. An acyclic C₂-C₁₀ hydrocarbon feedstock, useful herein, is obtainable from crude oil or natural gas condensate. Optionally, hydrogen may be present in the feedstock as well. The molar ratio of optional hydrogen to acyclic hydrocarbon is preferably between about 0 to about 3, or in the range of about 1 to about 2. Hydrogen may be included in the feedstock in order to minimize production of coke material on the particulate material and/or to fluidize the particulate material in the at least one reaction zone.

Preferably, in one or more embodiments, the acyclic C₅ feedstock comprises at least about 50 wt %, or 60 wt %, or 75 wt %, or 90 wt % acyclic hydrocarbons, or in the range from about 50 wt % to about 100 wt % n-pentane. Preferably, an amount of the acyclic hydrocarbons in the feedstock converted to alkenes (e.g., propylene), cyclic hydrocarbons (e.g., cyclopentadiene) and/or aromatics (e.g., benzene) is ≥about 5.0 wt %, ≥about 10.0 wt %, ≥about 20.0 wt %, ≥about 30.0 wt %, ≥about 40.0 wt %, ≥about 50.0 wt %, ≥about 60.0 wt %, ≥about 70.0 wt %, ≥about 80.0 wt %, or ≥about 90.0 wt %.

In various aspects, the feedstock may preferably be an acyclic C₅ feedstock and can include cracked C₅ (in various degrees of unsaturation: alkenes, dialkenes, alkynes) produced by refining and chemical processes, such as fluid catalytic cracking (FCC), reforming, hydrocracking, hydrotreating, coking, and steam cracking.

In one or more embodiments, the acyclic C₅ feedstock useful in the process of this invention comprises pentane, pentene, pentadiene and mixtures of two or more thereof. Preferably, in one or more embodiments, the acyclic C₅ feedstock comprises at least about 50 wt %, or 60 wt %, or 75 wt %, or 90 wt % n-pentane, or in the range from about 50 wt % to about 100 wt % n-pentane.

The acyclic hydrocarbon feedstock optionally does not comprise C₆ aromatic compounds, such as benzene. Preferably C₆ aromatic compounds are present at less than 5 wt %, preferably less than 1 wt %, preferably less than 0.01 wt %, and preferably at zero wt %. Additionally or alternatively, the acyclic hydrocarbon feedstock optionally does not comprise benzene, toluene, or xylene (ortho, meta, or para). Preferably any benzene, toluene, or xylene (ortho, meta, or para) compounds are present at less than 5 wt %, preferably less than 1 wt %, preferably less than 0.01 wt %, and preferably at zero wt %.

The acyclic hydrocarbon feedstock optionally does not comprise C₆₊ aromatic compounds. Preferably, C₆₊ aromatic compounds are present at less than 5 wt %, preferably less than 1 wt %, preferably less than 0.01 wt %, and preferably at zero wt %.

Preferably, an amount of the C₅ hydrocarbons (e.g., acyclic C₅ hydrocarbons) in the feedstock converted to cyclopentadiene is ≥about 5.0 wt %, ≥about 10.0 wt %, ≥about 20.0 wt %, ≥about 30.0 wt %, ≥about 40.0 wt %, ≥about 50.0 wt %, ≥about 60.0 wt %, ≥about 70.0 wt %, ≥about 80.0 wt %, or ≥about 90.0 wt %. Preferably, at least about 30.0 wt % or at least about 60.0 wt % of the C₅ hydrocarbons (e.g., acyclic C₅ hydrocarbons) is converted to cyclopentadiene. Ranges expressly disclosed include combinations of any of the above-enumerated values; e.g., about 5.0% to about 90.0 wt %, about 10.0 wt % to about 80.0 wt %, about 20.0 wt % to about 70.0 wt %, about 20.0 wt % to about 60.0 wt %, etc. Preferably, about 20.0 wt % to about 90.0 wt % of the C₅ hydrocarbons (e.g., acyclic C₅ hydrocarbons) is converted to cyclopentadiene, more preferably about 30.0 wt % to about 85.0 wt %, more preferably about 40.0 wt % to about 80.0 wt %, more preferably about 45.0 wt % to about 75.0 wt %, and more preferably about 50.0 wt % to about 70.0 wt %.

Preferably, a hydrogen co-feedstock comprising hydrogen and, optionally, light hydrocarbons, such as C₁-C₄ hydrocarbons, is also fed into the at least one reaction zone (discussed herein). Preferably, at least a portion of the hydrogen co-feedstock is admixed with the feedstock prior to being fed into the at least one reaction zone. The presence of hydrogen in the feed mixture at the inlet location, where the feed first comes into contact with the catalyst, prevents or reduces the formation of coke on the catalyst particles.

### B. Reaction Zone

The feedstock is fed into a reactor system and contacted with a catalyst material and an inert material in at least one reaction zone under reaction conditions to convert at least a portion of the acyclic hydrocarbons (e.g., acyclic C₅ hydrocarbons) to a first effluent comprising alkenes (e.g., propylene), cyclic hydrocarbons (e.g., cyclopentadiene) and aromatics (e.g., benzene). The at least one reaction zone may be a circulating fluidized bed reactor. As stated above, the catalyst material and the inert material preferably may have a different average diameter and/or density. Thus, due to the presence of catalyst material and the inert material having a different average diameter and/or density, the at least one reaction zone may operate in two fluidization regimes with respect to the individual group of catalyst and inert particles. For example, the circulating fluidized bed reactor may be operated in the bubbling or turbulent fluidization regimes and the fast fluidization or transport regime., both as described in Kunii, D., Levenspiel, O., Chapter 3 of Fluidization Engineering, 2^{nd} Edition, Butterworth-Heinemann, Boston, 1991 and Walas, S. M., Chapter 6 of Chemical Process Equipment, Revised 2^{nd} Edition, Butterworth-Heinemann, Boston, 2010. Additionally, or alternatively, the at least one reaction zone is not a radial-flow reactor or a cross-flow reactor.

Additionally, or alternatively, the at least one reaction zone may comprise at least a first reaction zone, a second reaction zone, a third reaction zone, a fourth reaction zone, a fifth reaction zone, a sixth reaction zone, a seventh reaction zone, and/or an eighth reaction zone, etc. As understood herein, each reaction zone may be an individual reactor or a reactor may comprise one or more of the reaction zones. Preferably, the reactor system includes 1 to 20 reaction zones, more preferably 1 to 15 reaction zones, more preferably 2 to 10 reaction zones, more preferably 2 to 8 reaction zones. Where the at least one reaction zone includes a first and a second reaction zone, the reaction zones may be arranged in any suitable configuration, preferably in series. Each reaction zone independently may be a circulating fluidized bed. Additionally, or alternatively, the process described herein may further comprise moving a bulk of a partially converted feedstock from the first reaction zone to the second reaction zone and/or moving a bulk of a particulate material (e.g., catalyst material and/or inert material) from the second reaction zone to the first reaction zone. As used herein, “bulk” refers to at least a majority portion of the partially converted feedstock and the particulate material, e.g., portions of at least about 50.0 wt %, at least about 60.0 wt %, at least about 70.0 wt %, at least about 80.0 wt %, at least about 90.0 wt %, at least about 95.0 wt %, at least about 99.0 wt %, and 100.0 wt %.

Preferably, the at least one reaction zone may include at least one internal structure, preferably a plurality of internal structures (e.g., 2, 3, 4, 5, 6, 7, 8, 9, 10, 15, 20, 30, 40, 50, etc.) to influence a velocity vector of the particulate material and/or gas flow. Further, the internal structure(s) can insure movement of particulate material while minimizing the degree of gas back-mixing. Particularly, the at least one reaction zone may include a plurality of internal structures. Examples of suitable internal structures include a plurality of baffles, sheds, trays, tubes, tube bundles, tube coils, rods, and/or distributors.

The at least one reaction zone is operated under reaction conditions sufficient to convert at least a portion of the acyclic hydrocarbons feedstock, preferably acyclic C₅ hydrocarbons, to a first effluent comprising alkene, cyclic hydrocarbons, and aromatics, preferably cyclopentadiene. Preferably, the feedstock (e.g., acyclic hydrocarbons) may be fed to the reaction system at a weight hourly space velocity (WHSV, mass of acyclic hydrocarbons/mass of catalyst/hour) in the range of from about 1.0 to about 1000.0 hr⁻¹. The WHSV may be about 1.0 to about 900.0 hr⁻¹, about 1.0 to about 800.0 hr⁻¹, about 1.0 to about 700.0 hr⁻¹, about 1.0 to about 600.0 hr⁻¹, about 1.0 to about 500.0 hr⁻¹, about 1.0 to about 400.0 hr⁻¹, about 1.0 to about 300.0 hr⁻¹, about 1.0 to about 200.0 hr⁻¹, about 1.0 to about 100.0 hr⁻¹, about 1.0 to about 90.0 hr⁻¹, about 1.0 to about 80.0 hr⁻¹, about 1.0 to about 70.0 hr⁻¹, about 1.0 to about 60.0 hr⁻¹, about 1.0 to about 50.0 hr⁻¹, about 1.0 to about 40.0 hr⁻¹, about 1.0 to about 30.0 hr⁻¹, about 1.0 to about 20.0 hr⁻¹, about 1.0 to about 10.0 hr⁻¹, about 1.0 to about 5.0 hr⁻¹, about 2.0 to about 1000.0 hr⁻¹, about 2.0 to about 900.0 hr⁻¹, about 2.0 to about 800.0 hr⁻¹, about 2.0 to about 700.0 hr⁻¹, about 2.0 to about 600.0 hr⁻¹, about 2.0 to about 500.0 hr⁻¹, about 2.0 to about 400.0 hr⁻¹, about 2.0 to about 300.0 hr⁻¹, about 2.0 to about 200.0 hr⁻¹, about 2.0 to about 100.0 hr⁻¹, about 2.0 to about 90.0 hr⁻¹, about 2.0 to about 80.0 hr⁻¹, about 2.0 to about 70.0 hr⁻¹, about 2.0 to about 60.0 hr⁻¹, about 2.0 to about 50.0 hr⁻¹, about 2.0 to about 40.0 hr⁻¹, about 2.0 to about 30.0 hr⁻¹, about 2.0 to about 20.0 hr⁻¹, about 2.0 to about 10.0 hr⁻¹, and about 2.0 to about 5.0 hr⁻¹. Preferably, the WHSV is about 1.0 to about 100.0 hr⁻¹, more preferably about 1.0 to about 60.0 hr⁻¹, more preferably about 2.0 to about 40.0 hr⁻¹, more preferably about 2.0 to about 20.0 hr⁻¹.

Additionally, it may be preferable that an isothermal or substantially isothermal temperature profile be maintained in the at least one reaction zone. A substantially isothermal temperature profile has the advantages of maximizing the effective utilization of the catalyst and minimizing the production of undesirable C4− byproducts. As used herein, “isothermal temperature profile” means that the temperature at each point within the reaction zone between the reactor inlet and reactor outlet as measured along the tube centerline of the reactor is kept essentially constant, e.g., at the same temperature or within the same narrow temperature range wherein the difference between an upper temperature and a lower temperature is no more than about 40° C.; more preferably no more than about 20° C. Preferably, the isothermal temperature profile is one where the temperature along the length of the reaction zone(s) within the reactor does not vary by more than about 40° C. as compared to the average temperature within the reactor, alternately not more than about 20° C., alternately not more than about 10° C., alternately not more than about 5° C. Alternately, the isothermal temperature profile is one where the temperature along the length of the reaction zone(s) within the reactor is within about 20% of the average temperature within the reactor, alternately within about 10%, alternately within about 5%, alternately within about 1% of the average temperature within the reactor.

Thus, the temperature of the feedstock (e.g., acyclic hydrocarbons) entering the reactor system at a feedstock inlet may be ≤about 750° C., ≤about 725° C., ≤about 700° C., ≤about 675° C., ≤about 650° C., ≤about 625° C., ≤about 600° C., ≤about 575° C., ≤about 550° C., ≤about 525° C., ≤about 500° C., ≤about 475° C., ≤about 450° C., ≤about 425° C., ≤about 400° C., ≤about 375° C., ≤about 350° C., ≤about 325° C., ≤or about 300° C. Preferably, the temperature of the feedstock (e.g., acyclic hydrocarbons) entering the reactor system is ≤about 675° C., more preferably ≤about 650° C., or more preferably ≤about 625° C. Ranges of temperatures expressly disclosed include combinations of any of the above-enumerated values, e.g., about 300° C. to about 750° C., about 350° C. to about 700° C., about 450° C. to about 650° C., about 475° C. to about 600° C., etc. Preferably, the temperature of the feedstock (e.g., acyclic hydrocarbons) entering the reaction system is about 300° C. to about 750° C., more preferably about 300° C. to about 700° C., more preferably about 400° C. to about 700° C., and more preferably about 450° C. to about 600° C. Providing the feedstock (e.g., acyclic C₅ hydrocarbons) at the above-described temperatures may advantageously minimize undesirable cracking of the C₅ hydrocarbons (e.g., acyclic C₅ hydrocarbons) before they can react in the presence of the catalyst material. The feedstock may be heated, optionally in the presence a hydrogen co-feed, in a fired-tube furnace and/or heat exchanger to achieve the above-described temperatures before entering the at least one reaction zone.

Additionally, the temperature of a first effluent exiting the at least one reaction zone at an effluent outlet may be ≥about 400° C., ≥about 425° C., ≥about 450° C., ≥about 475° C., ≥about 500° C., ≥about 525° C., ≥about 550° C., ≥about 575° C., ≥about 600° C., ≥about 625° C., ≥about 650° C., ≥about 675° C., or ≥about 700° C. Preferably, the temperature of a first effluent exiting the at least one reaction zone is ≥about 550° C., more preferably ≥about 575° C., more preferably ≥about 600° C. Ranges of temperatures expressly disclosed include combinations of any of the above-enumerated values, e.g., about 400° C. to about 700° C., about 475° C. to about 675° C., about 525° C. to about 650° C., about 550° C. to about 600° C., etc. Preferably, the temperature of a first effluent exiting the at least one reaction zone is about 475° C. to about 700° C., more preferably about 500° C. to about 650° C., more preferably about 550° C. to about 625° C.

Additionally, or alternatively, reaction conditions in the at least one reaction zone may include a temperature of ≥about 300° C., ≥about 325° C., ≥about 350° C., ≥about 375° C., ≥about 400° C., ≥about 425° C., ≥about 450° C., ≥about 475° C., ≥about 500° C., ≥about 525° C., ≥about 550° C., ≥about 575° C., ≥about 600° C. ≥about 625° C., ≥about 650° C., ≥about 675° C., or ≥about 700° C. Ranges of temperatures expressly disclosed include combinations of any of the above-enumerated values, e.g., about 300° C. to about 700° C., about 350° C. to about 675° C., and about 400° C. to about 700° C., etc. Preferably, the temperature may be about 350° C. to about 700° C., more preferably about 400° C. to about 700° C., or more preferably about 500° C. to about 650° C. Optionally, the at least one reaction zone may include one or more heating devices in order to maintain a temperature therein. Examples of suitable heating devices known in the art include, but are not limited to a fired tube, a heated coil with a high temperature heat transfer fluid, an electrical heater, and/or a microwave emitter. As used herein, “coil” refers to a structure placed within a vessel through which a heat transfer fluid flows to transfer heat to the vessel contents. A coil may have any suitable cross-sectional shape and may be straight, include u-bends, include loops, etc.

Additionally, or alternatively, reaction conditions at the effluent outlet of the at least one reaction zone may include a pressure of ≥about 1.0 psia, ≥about 2.0 psia, ≥about 3.0 psia, ≥about 4.0 psia, ≥about 5.0 psia, ≥about 10.0 psia, ≥about 15.0 psia, ≥about 20.0 psia, ≥about 25.0 psia, ≥about 30.0 psia, ≥about 35.0 psia, ≥about 40.0 psia, ≥about 45.0 psia, ≥about 50.0 psia, ≥about 55.0 psia, ≥about 60.0 psia, ≥about 65.0 psia, ≥about 70.0 psia, ≥about 75.0 psia, ≥about 80.0 psia, ≥about 85.0 psia, ≥about 90.0 psia, ≥about 95.0 psia, ≥about 100.0 psia, ≥about 125.0 psia, ≥about 150.0 psia, ≥about 175.0 psia or about 200 psia. Ranges and combinations of temperatures and pressures expressly disclosed include combinations of any of the above-enumerated values, e.g., about 1.0 psia to about 200.0 psia, about 2.0 psia to about 175.0 psia, about 5.0 psia to about 95.0 psia, etc. Preferably, the pressure may be about 3.0 psia to about 100.0 psia, more preferably about 3.0 psia to about 50.0 psia, more preferably about 3.0 psia to about 20.0 psia. In particular, the reaction conditions may comprise a temperature of about 400° C. to about 700° C. and a pressure of about 3.0 psia to about 100 psia.

Additionally, or alternatively, a delta pressure across the at least one reaction zone (pressure at feedstock inlet minus pressure at effluent outlet) may be ≥about 0.5 psia, ≥about 1.0 psia, ≥about 2.0 psia, ≥about 3.0 psia, ≥about 4.0 psia, ≥about 5.0 psia, ≥about 10.0 psia, ≥about 14.0 psia, ≥about 15.0, psia, ≥about 20.0 psia, ≥about 24.0 psia, ≥about 25.0 psia, ≥about 30.0 psia, ≥about 35.0 psia, ≥about 40.0 psia, ≥about 45.0 psia, ≥about 50.0 psia, ≥about 55.0 psia, ≥about 60.0 psia, ≥about 65.0 psia, ≥about 70.0 psia, ≥about 75.0 psia, ≥about 80.0 psia, ≥about 85.0 psia, ≥about 90.0 psia, ≥about 95.0 psia, ≥about 100.0 psia, ≥about 125.0 psia, or ≥about 150.0 psia. As understood herein, “at a feedstock inlet,” “at an inlet,” “at an effluent outlet,” and “at an outlet” includes the space in and substantially around the inlet and/or outlet. Additionally, or alternatively, a delta pressure (or pressure drop) across the at least one reaction zone (pressure at feedstock inlet minus pressure at effluent outlet) may be ≤about 2.0 psia, ≤about 3.0 psia, ≤about 4.0 psia, ≤about 5.0 psia, ≤about 10.0 psia, ≤about 14.0 psia, ≤about 15.0 psia, ≤about 20.0 psia, ≤about 24.0 psia, ≤about 25.0 psia, ≤about 30.0 psia, ≤about 35.0 psia, ≤about 40.0 psia, ≤about 45.0 psia, ≤about 50.0 psia, ≤about 55.0 psia, ≤about 60.0 psia, ≤about 65.0 psia, ≤about 70.0 psia, ≤about 75.0 psia, ≤about 80.0 psia, ≤about 85.0 psia, ≤about 90.0 psia, ≤about 95.0 psia, ≤about 100.0 psia, ≤about 125.0 psia, ≤about 150.0 psia, ≤about 175.0 psia, or ≤about 200.0 psia. Ranges of delta pressures expressly disclosed include combinations of any of the above-enumerated values, e.g., about 10 psia to about 70.0 psia, about 20.0 psia to about 60.0 psia, about 30.0 psia to about 50.0 psia, etc. In particular, the pressure substantially at an inlet of a feedstock (e.g., acyclic C₅ hydrocarbons) may be about 10.0 psia to about 70.0 psia, preferably about 10.0 psia to about 60.0 psia, more preferably about 10.0 psia to about 40.0 psia. Additionally, the pressure substantially at an outlet of at least a first effluent may be about 1.0 psia to about 60.0 psia, preferably about 5.0 psia to about 40.0 psia, more preferably about 10.0 psia to about 30.0 psia.

Additionally or alternatively, a stream comprising hydrogen may be fed to the at least one reaction zone. Such a stream comprising hydrogen may be introduced into the at least one reaction zone in order to minimize production of coke material on the particulate material and/or to fluidize the particulate material in the at least one reaction zone. Such a stream comprising hydrogen may contain light hydrocarbons (e.g., C₁-C₄); preferably the content of light hydrocarbons is less than about 50 mol %, less than about 40 mol %, less than about 30 mol %, less than about 20 mol %, less than about 10 mol %, less than about 5 mol %, less than about 1 mol %. Preferably, the stream comprising hydrogen is substantially free of oxygen, e.g., less than about 1.0 wt %, less than about 0.1 wt %, less than about 0.01 wt %, less than about 0.001 wt %, less than about 0.0001 wt %, less than about 0.00001 wt %, etc.

### C. Catalyst Material and Inert Material

The at least one reaction zone comprises particulate material including a catalyst material and an inert material. The catalyst material, also referred to as a “catalyst composition,” is present in the reaction system for promoting conversion of at least a portion of the acyclic hydrocarbons to alkenes, cyclic hydrocarbons and aromatics, in particular conversion of acyclic C₅ hydrocarbons to cyclopentadiene.

Catalyst compositions useful herein include microporous crystalline metallosilicates, such as crystalline aluminosilicates, crystalline ferrosilicates, or other metal-containing crystalline silicates (such as those where the metal or metal-containing compound is dispersed within the crystalline silicate structure and may or may not be a part of the crystalline framework). Microporous crystalline metallosilicate framework types useful as catalyst compositions herein include, but are not limited to, MWW, MFI, LTL, MOR, BEA, TON, MTW, MTT, FER, MRE, MFS, MEL, DDR, EUO, and FAU.

Particularly suitable microporous metallosilicates for use herein, include those of framework type MWW, MFI, LTL, MOR, BEA, TON, MTW, MTT, FER, MRE, MFS, MEL, DDR, EUO, and FAU (such as zeolite beta, mordenite, faujasite, Zeolite L, ZSM-5, ZSM-11, ZSM-22, ZSM-23, ZSM-35, ZSM-48, ZSM-50, ZSM-57, ZSM-58, and MCM-22 family materials) where one or more metals from groups 8, 11, and 13 of the Periodic Table of the Elements (preferably one or more of Fe, Cu, Ag, Au, B, Al, Ga, and/or In) are incorporated in the crystal structure during synthesis or impregnated post crystallization. It is recognized that a metallosilicate may have one of more metals present and, for example, a material may be referred to as a ferrosilicate, but it will most likely still contain small amounts of aluminum.

The microporous crystalline metallosilicates preferably have a constraint index of less than 12, alternately from 1 to 12, alternately from 3 to 12. Aluminosilicates useful herein have a constraint index of less than 12, such as 1 to 12, alternately 3 to 12, and include, but are not limited to Zeolite beta, mordenite, faujasite, Zeolite L, ZSM-5, ZSM-11, ZSM-22, ZSM-23, ZSM-35, ZSM-48, ZSM-50, ZSM-57, ZSM-58, MCM-22 family materials, and mixtures of two or more thereof. In a preferred embodiment, the crystalline aluminosilicate has a constraint index of about 3 to about 12 and is ZSM-5.

ZSM-5 is described in U.S. Pat. No. 3,702,886. ZSM-11 is described in U.S. Pat. No. 3,709,979. ZSM-22 is described in U.S. Pat. No. 5,336,478. ZSM-23 is described in U.S. Pat. No. 4,076,842. ZSM-35 is described in U.S. Pat. No. 4,016,245. ZSM-48 is described in U.S. Pat. No. 4,375,573. ZSM-50 is described in U.S. Pat. No. 4,640,829. ZSM-57 is described in U.S. Pat. No. 4,873,067. ZSM-58 is described in U.S. Pat. No. 4,698,217.

The MCM-22 family material is selected from the group consisting of MCM-22, PSH-3, SSZ-25, MCM-36, MCM-49, MCM-56, ERB-1, EMM-10, EMM-10-P, EMM-12, EMM-13, UZM-8, UZM-8HS, ITQ-1, ITQ-2, ITQ-30, and mixtures of two or more thereof.

Materials of the MCM-22 family include MCM-22 (described in U.S. Pat. No. 4,954,325), PSH-3 (described in U.S. Pat. No. 4,439,409), SSZ-25 (described in U.S. Pat. No. 4,826,667), ERB-1 (described in EP 0 293 032), ITQ-1 (described in U.S. Pat. No. 6,077,498), and ITQ-2 (described in WO 97/17290), MCM-36 (described in U.S. Pat. No. 5,250,277), MCM-49 (described in U.S. Pat. No. 5,236,575), MCM-56 (described in U.S. Pat. No. 5,362,697), and mixtures of two or more thereof. Related zeolites to be included in the MCM-22 family are UZM-8 (described in U.S. Pat. No. 6,756,030) and UZM-8HS (described in U.S. Pat. No. 7,713,513), both of which are also suitable for use as the molecular sieve of the MCM-22 family.

In one or more embodiments, the crystalline metallosilicate has an Si/M molar ratio (where M is a group 8, 11, or 13 metal) greater than about 3, or greater than about 25, or greater than about 50, or greater than about 100, or greater than 400, or in the range from about 100 to about 2,000, or from about 100 to about 1,500, or from about 50 to 2,000, or from about 50 to 1,200.

In one or more embodiments, the crystalline aluminosilicate has an SiO₂/Al₂O₃ molar ratio greater than about 3, or greater than about 25, or greater than about 50, or greater than about 100, or greater than about 400, or greater than about 1,000, or in the range from about 100 to about 400, or from about 100 to about 500, or from about 25 to about 2,000, or from about 50 to about 1,500, or from about 100 to about 1,200, or from about 50 to about 1,000.

Typically, the microporous crystalline metallosilicate (such as an aluminosilicate) is combined with a Group 10 metal or metal compound and, optionally, one, two, three, or more additional metals selected from Groups 8, 9, 11, and 13 of the Periodic Table of the Elements and the rare earth metals, such as Ga, In, Zn, Cu, Re, Mo, W, La, Fe, Ag, Rh, Pr, La, and/or oxides, sulfides, nitrides, and/or carbides of these metals. Alternatively or additionally, the Group 10 metal is present in combination with a Group I alkali metal and/or a Group 2 alkaline earth metal.

In one or more embodiments, the Group 10 metal includes, or is selected from the group consisting of, Ni, Pd, and Pt, preferably Pt. The Group 10 metal content of said catalyst composition is at least 0.005 wt %, based on the weight of the catalyst composition. In one or more embodiments, the Group 10 content is in the range from about 0.005 wt % to about 10 wt %, or from about 0.005 wt % up to about 1.5 wt %, based on the weight of the catalyst composition.

The Group 1 alkali metal is generally present as an oxide and the metal is selected from the group consisting of lithium, sodium, potassium, rubidium, cesium, and mixtures of two or more thereof. The Group 2 alkaline earth metal is generally present as an oxide and the metal is selected from the group consisting of beryllium, magnesium, calcium, strontium, barium, and mixtures of two or more thereof.

In one or more embodiments, the Group 11 metal includes, or is selected from the group consisting of, silver, gold, copper, preferably silver or copper. The Group 11 metal content of said catalyst composition is at least 0.005 wt %, based on the weight of the catalyst composition. In one or more embodiments, the Group 11 content is in the range from about 0.005 wt % to about 10 wt %, or from about 0.005 wt % up to about 1.5 wt %, based on the weight of the catalyst composition. In one or more embodiments, the molar ratio of said Group 11 metal to Group 10 metal is at least about 0.1, or from at least about 0.1 up to about 10, preferably at least about 0.5, more preferably at least about 1. In one or more embodiments, the Group 11 metal is present as an oxide.

A preferred Group 9 metal is Rh, which may form an alloy with the Group 10 metal. Preferably, the molar ratio of Rh to Group 10 metal is in the range from about 0.1 to about 5.

Typically, the rare earth metal is selected from the group consisting of yttrium, lanthanum, cerium, praseodymium, and mixtures or combinations thereof. Preferably, the molar ratio of rare earth metal to Group 10 metal is in the range from about 1 to about 10. The rare earth metal may be added to the catalyst composition during or after synthesis of the microporous crystalline molecular sieve as any suitable rare earth metal compound.

In one or more embodiments of aluminosilicates, the molar ratio of said Group 1 alkali metal to Al is at least about 0.5, or from at least about 0.5 up to about 3, preferably at least about 1, more preferably at least about 2.

In one or more embodiments of aluminosilicates, the molar ratio of said Group 2 alkaline earth metal to Al is at least about 0.5, or from at least about 0.5 up to about 3, preferably at least about 1, more preferably at least about 2.

In one or more embodiments, the catalyst composition has an Alpha Value (as measured prior to the addition of the Group 10 metal, preferably platinum) of less than 25, alternately less than 15, alternately from 1 to 25, alternately from 1.1 to 15. Alpha Value is determined as described in U.S. Pat. No. 3,354,078; The Journal of Catalysis, v. 4, p. 527 (1965); v. 6, p. 278 (1966); and v. 61, p. 395 (1980) using a constant temperature of 538° C. and a variable flow rate, as described in detail in The Journal of Catalysis, v. 61, p. 395, (1980).

In one or more embodiments, the use of any one of the catalyst compositions of this invention provides a conversion of at least about 70%, or at least about 75%, or at least about 80%, or in the range from about 60% to about 80%, of said acyclic C₅ feedstock under acyclic C₅ conversion conditions. This includes an n-pentane containing feedstock with equimolar H₂, a temperature in the range of about 550° C. to about 600° C., an n-pentane partial pressure between 3 and 10 psia, and an n-pentane weight hourly space velocity of 10 to 20 hr¹.

In one or more embodiments, the use of any one of the catalyst compositions of this invention provides a carbon selectivity to cyclic C₅ compounds of at least about 30%, or at least about 40%, or at least about 50%, or in the range from about 30% to about 80%, under acyclic C₅ conversion conditions. This includes an n-pentane feedstock with equimolar H₂, a temperature in the range of about 550° C. to about 600° C., an n-pentane partial pressure between 3 and 10 psia, and an n-pentane weight hourly space velocity between 10 and 20 hr⁻¹.

In one or more embodiments, the use of any one of the catalyst compositions of this invention provides a carbon selectivity to cyclopentadiene of at least about 30%, or at least about 40%, or at least about 50%, or in the range from about 30% to about 80%, under acyclic C₅ conversion conditions. This includes an n-pentane feedstock with equimolar H₂, a temperature in the range of about 550° C. to about 600° C., an n-pentane partial pressure between 3 and 10 psia, and an n-pentane weight hourly space velocity between 10 and 20 hr⁻¹.

The catalyst compositions of this invention can be combined with a matrix or binder material to render them attrition resistant and more resistant to the severity of the conditions to which they will be exposed during use in hydrocarbon conversion applications. Preferred binder materials comprise one or more of silica, titania, zirconia, metal silicates of Group 1 or Group 13 of the Periodic Table, carbides, nitrides, aluminum phosphate, aluminum molybdate, aluminate, surface passivated alumina, and mixtures thereof. Preferably, suitable binder materials have a lower affinity for Group 10 metal particles, e.g. Pt, in comparison with the crystalline metallosilicate, e.g. aluminosilicate. The combined compositions can contain 1 to 99 wt % of the materials of the invention based on the combined weight of the matrix (binder) and material of the invention. The relative proportions of microcrystalline material and matrix may vary widely, with the crystal content ranging from about 1 to about 90 wt % and, more usually, particularly when the composite is prepared in the form of beads, extrudates, pills, oil drop formed particles, spray dried particles, etc., in the range of about 2 to about 80 wt % of the composite.

Useful catalyst compositions comprise a crystalline aluminosilicate or ferrosilicate, which is optionally combined with one, two, or more additional metals or metal compounds. Preferred combinations include:

1) a crystalline aluminosilicate (such as ZSM-5 or Zeolite L) combined with a Group 10 metal (such as Pt), a Group 1 alkali metal (such as sodium or potassium) and/or a Group 2 alkaline earth metal;

2) a crystalline aluminosilicate (such as ZSM-5 or Zeolite L) combined with a Group 10 metal (such as Pt), and a Group 1 alkali metal (such as sodium or potassium);

3) a crystalline aluminosilicate (such as a ferrosilicate or an iron treated ZSM-5) combined with a Group 10 metal (such as Pt) and a Group 1 alkali metal (such as sodium or potassium);

4) a crystalline aluminosilicate (Zeolite L) combined with a Group 10 metal (such as Pt) and a Group 1 alkali metal (such as potassium); and
5) a crystalline aluminosilicate (such as ZSM-5) combined with a Group 10 metal (such as Pt), a Group 1 alkali metal (such as sodium), and a Group 11 metal (such as silver or copper).

Another useful catalyst composition is a Group 10 metal (such as Ni, Pd, and Pt, preferably Pt) supported on silica (e.g., silicon dioxide) modified by a Group 1 alkali metal silicate (such as Li, Na, K, Rb, and/or C₅ silicates) and/or a Group 2 alkaline earth metal silicate (such as Mg, Ca, Sr, and/or Ba silicates), preferably potassium silicate, sodium silicate, calcium silicate, and/or magnesium silicate, preferably potassium silicate and/or sodium silicate. The Group 10 metal content of the catalyst composition is at least 0.005 wt %, based on the weight of the catalyst composition, preferably, in the range from about 0.005 wt % to about 10 wt %, or from about 0.005 wt % up to about 1.5 wt %, based on the weight of the catalyst composition. The silica (SiO₂) may be any silica typically used as catalyst support such as those marketed under the tradenames of DAVISIL 646 (Sigma Aldrich), DAVISON 952, DAVISON 948 or DAVISON 955 (Davison Chemical Division of W.R. Grace and Company).

Catalyst composition shape and design are preferably configured to minimize pressure drop, increase heat transfer, and minimize mass transport phenomena. Suitable catalyst shape and design are described in WO 2014/053553, which is incorporated by reference in its entirety. The catalyst composition may be an extrudate with a diameter of 2 mm to 20 mm Optionally, the catalyst composition cross section may be shaped with one or more lobes and/or concave sections. Additionally, the catalyst composition lobes and/or concave sections may be spiraled. The catalyst composition may be an extrudate with a diameter of 2 mm to 20 mm; and the catalyst composition cross section may be shaped with one or more lobes and/or concave sections; and the catalyst composition lobes and/or concave sections may be spiraled. Also, the formulated catalyst composition may be made into a particle, such as, for example, a spray dried particle, an oil drop particle, a mulled particle, or a spherical particle. The formulated catalyst composition may be made into a slurry. Such slurry materials typically contain the microporous crystalline metallosilicate, such as zeolite, and a filler such as a silicate. For fluid bed reactors spherical particle shapes are particularly useful.

For more information on useful catalyst compositions, please see applications: U.S. Ser. No. 62/250,675, filed Nov. 4, 2015; U.S. Ser. No. 62/250,681, filed Nov. 4, 2015; U.S. Ser. No. 62/250,688, filed Nov. 4, 2015; U.S. Ser. No. 62/250,695, filed Nov. 4, 2015; and U.S. Ser. No. 62/250,689, filed Nov. 4, 2015; which are incorporated herein by reference.

Preferably, the catalyst material comprises platinum on ZSM-5, platinum on zeolite L, and/or platinum on silica.

Suitable amounts of catalyst material in the particulate material may be ≤about 1.0 wt %, ≤about 5.0 wt %, ≤about 10.0 wt %, ≤about 15.0 wt %, ≤about 20.0 wt %, ≤about 25.0 wt %, ≤about 30.0 wt %, ≤about 35.0 wt %, ≤about 40.0 wt %, ≤about 45.0 wt %, ≤about 50.0 wt %, ≤55.0 wt %, ≤about 60.0 wt %, ≤about 65.0 wt %, ≤about 70.0 wt %, ≤about 75.0 wt %, ≤about 80.0 wt %, ≤about 85.0 wt %, ≤about 90.0 wt %, ≤about 95.0 wt %, ≤about 99.0 wt % or about 100.0 wt %. Preferably, the particulate material may comprise ≤about 75.0 wt % catalyst material. Additionally, or alternatively, the particulate material may comprise the catalyst material in an amount of ≥about 1.0 wt %, ≥about 5.0 wt %, ≥about 10.0 wt %, ≥about 15.0 wt %, ≥about 20.0 wt %, ≥about 25.0 wt %, ≥about 30.0 wt %, ≥about 35.0 wt %, ≥about 40.0 wt %, ≥about 45.0 wt %, ≥about 50.0 wt %, ≥about 55.0 wt %, ≥about 60.0 wt %, ≥about 65.0 wt %, ≥about 70.0 wt %, ≥about 75.0 wt %, ≥about 80.0 wt %, ≥about 85.0 wt %, ≥about 90.0 wt %, or ≥about 95.0 wt %. Ranges expressly disclosed include combinations of any of the above-enumerated values; e.g., about 1.0 wt % to about 100.0 wt %, about 5.0 wt % to about 100.0 wt %, about 10.0 wt % to about 90.0 wt %, about 20.0 wt % to about 80.0 wt %, etc. Preferably, the particulate material may comprise the catalyst material in an amount of about 5.0 wt % to about 90.0 wt %, more preferably about 10.0 wt % to about 80.0 wt %, more preferably about 20.0 wt % to about 70.0 wt %, more preferably about 30.0 wt % to about 60.0 wt %.

In addition to the catalyst material, inert material may also be present in the at least one reaction zone. As referred to herein, the inert material is understood to include materials which promote a negligible amount (e.g., ≤about 3%, ≤about 2%, ≤about 1%, etc.) of conversion of the feedstock, intermediate products, or final products under the reaction conditions described herein. The catalyst material and the inert material may be combined as portions of the same particles and/or may be separate particles. Preferably the catalyst material and the inert material are separate particles. Additionally, the catalyst material and/or inert material may be essentially spherical (i.e., ≤about 20%, ≤about 30%, ≤about 40%, or ≤about 50% aberration in diameter). Examples of suitable inert materials include, but are not limited to metal carbides (e.g., silicon carbide, tungsten carbide, etc.), metal oxides (e.g., silica, zirconia, titania, alumina, etc.), clays, metal phosphates (e.g., aluminum phosphates, nickel phosphates, zirconium phosphates, etc.), and combinations thereof. In particular, the inert material may comprise silicon carbide, silica, and a combination thereof.

Suitable amounts of inert material in the particulate material may be about 0.0 wt %, ≥about 1.0 wt %, ≥about 5.0 wt %, ≥about 10.0 wt %, ≥about 15.0 wt %, ≥about 20.0 wt %, ≥about 25.0 wt %, ≥about 30.0 wt %, ≥about 35.0 wt %, ≥about 40.0 wt %, ≥about 45.0 wt %, ≥about 50.0 wt %, ≥about 55.0 wt %, ≥about 60.0 wt %, ≥about 65.0 wt %, ≥about 70.0 wt %, ≥about 75.0 wt %, ≥about 80.0 wt %, ≥about 85.0 wt %, ≥about 90.0 wt %, ≥about 95.0 wt %, or ≥about 99.0 wt %. Preferably, the particulate material may comprise ≥about 25.0 wt % inert material. Additionally, or alternatively, the particulate material may comprise an inert material in an amount of ≤about 1.0 wt %, ≤about 5.0 wt %, ≤about 10.0 wt %, ≤about 15.0 wt %, ≤about 20.0 wt %, ≤about 25.0 wt %, ≤about 30.0 wt %, ≤about 35.0 wt %, ≤about 40.0 wt %, ≤about 45.0 wt %, ≤about 50.0 wt %, ≤about 55.0 wt %, ≤about 60.0 wt %, ≤about 65.0 wt %, ≤about 70.0 wt %, ≤about 75.0 wt %, ≤about 80.0 wt %, ≤about 85.0 wt %, ≤about 90.0 wt %, ≤about 95.0 wt %, or ≤about 99.0 wt %. Ranges expressly disclosed include combinations of any of the above-enumerated values, e.g., about 0.0 wt % to about 99.0 wt %, about 0.0 wt % to about 95.0 wt %, about 10.0 wt % to about 90.0 wt %, about 20.0 wt % to about 80.0 wt %, etc. Preferably, the particulate material may comprise an inert material in an amount of about 0.0 wt % to about 95.0 wt %, more preferably about 10 wt % to about 90 wt %, more preferably about 30.0 wt % to about 80.0 wt %, more preferably about 40.0 wt % to about 70.0 wt %.

In various aspects, the catalyst material and/or the inert material may have an average diameter of ≥about 25 μm, ≥about 50 μm, ≥about 100 μm, ≥about 200 μm, ≥about 300 μm, ≥about 400 μm, ≥about 500 μm, ≥about 600 μm, ≥about 700 μm, ≥about 800 μm, ≥about 900 μm, ≥about 1000 μm, ≥about 1100 μm, ≥about 1200 μm, ≥about 1300 μm, ≥about 1400 μm, ≥about 1500 μm, ≥about 1600 μm, ≥about 1700 μm, ≥about 1800 μm, ≥about 1900 μm, ≥about 2000 μm, ≥about 2100 μm, ≥about 2200 μm, ≥about 2300 μm, ≥about 2400 μm, ≥about 2500 μm, ≥about 2600 μm, ≥about 2700 μm, ≥about 2800 μm, ≥about 2900 μm, ≥about 3000 μm, ≥about 3100 μm, ≥about 3200 μm, ≥about 3300 μm, ≥about 3400 μm, ≥about 3500 μm, ≥about 3600 μm, ≥about 3700 μm, ≥about 3800 μm, ≥about 3900 μm, ≥about 4000 μm, ≥about 4100 μm, ≥about 4200 μm, ≥about 4300 μm, ≥about 4400 μm, ≥about 4500 μm, ≥about 5000 μm, ≥about 5500 μm, ≥about 6000 μm, ≥about 6500 μm, ≥about 7000 μm, ≥about 7500 μm, ≥about 8000 μm, ≥about 8500 μm, ≥about 9000 μm, ≥about 9500 μm, or ≥about 10,000 μm. Additionally, or alternatively, the catalyst material and/or the inert material may have an average diameter of ≤about 50 μm, ≤about 100 μm, ≤about 200 μm, ≤about 300 μm, ≤about 400 μm, ≤about 500 μm, ≤about 600 μm, ≤about 700 μm, ≤about 800 μm, ≤about 900 μm, ≤about 1000 μm, ≤about 1100 μm, ≤about 1200 μm, ≤about 1300 μm, ≤about 1400 μm, ≤about 1500 μm, ≤about 1600 μm, ≤about 1700 μm, ≤about 1800 μm, ≤about 1900 μm, ≤about 2000 μm, ≤about 2100 μm, ≤about 2200 μm, ≤about 2300 μm, ≤about 2400 μm, ≤about 2500 μm, ≤about 2600 μm, ≤about 2700 μm, ≤about 2800 μm, ≤about 2900 μm, ≤about 3000 μm, ≤about 3100 μm, ≤about 3200 μm, ≤about 3300 μm, ≤about 3400 μm, ≤about 3500 μm, ≤about 3600 μm, ≤about 3700 μm, ≤about 3800 μm, ≤about 3900 μm, ≤about 4000 μm, ≤about 4100 μm, ≤about 4200 μm, ≤about 4300 μm, ≤about 4400 μm, ≤about 4500 μm, ≤about 5000 μm, ≤about 5500 μm, ≤about 6000 μm, ≤about 6500 μm, ≤about 7000 μm, ≤about 7500 μm, ≤about 8000 μm, ≤about 8500 μm, ≤about 9000 μm, ≤about 9500 μm, or ≤about 10,000 μm. Ranges expressly disclosed include combinations of any of the above-enumerated values, e.g., about 25 μm to about 10,000 μm, about 50 μm to about 10,000 μm, about 100 μm to about 9000 μm, about 200 μm to about 7500 μm, about 200 μm to about 5500 μm, about 100 μm to about 4000 μm, about 100 μm to about 700 μm, etc. Preferably, in a circulating fluidized bed, the catalyst material and/or the inert material may have an average diameter of about 20 μm to about 1000 μm, more preferably about 20 μm to about 300 μm, more preferably about 20 μm to about 100 μm, more preferably about 40 μm to about 90 μm.

The at least one reaction zone is preferably bimodal, that is the catalyst material and the inert material preferably have a different average particle diameter and/or different density such that the at least one reaction can operate in two fluidization regimes with respect to the individual group of catalyst and inert particles. In various aspects, the catalyst material may have an average particle diameter and/or density greater than an average particle diameter and/or density of the inert material. Alternatively, the inert material may have an average particle diameter and/or density greater than an average particle diameter and/or density of the catalyst material.

Additionally, or alternatively, the difference in average particle diameter and/or density between the catalyst material and the inert material may be understood in terms of comparing a fluidization index of the catalyst material particles and a fluidization index of the inert material particles. A particle's fluidization index can be calculated according to equation (1) below:

Fluidization Index=ρₚ*dₚ² (1)

where ρₚ is particle density and dₚ is particle diameter.

In various aspects, the particulate material (e.g., catalyst material and inert material) in the at least one reaction zone may have the following relationship as shown in equation (2):

$$\frac{{( Fluidization Index )}_{particle 1}}{{( Fluidization Index )}_{particle 2}} < n \qquad ( 2 )$$

wherein “particle 1” and “particle 2” may be the catalyst material particle or the inert material particle, provided that particle 1 and particle 2 are different and the (Fluidization Index)_{particle 1} is <(Fluidization Index)_{particle 2}. Further, “n” may be <about 1, preferably <about 0.8 and more preferably <about 0.5. Preferably, particle 1 may be the catalyst material particle.

In one embodiment, the (Fluidization Index)_{catalyst particle} can be <the (Fluidization Index)_{inert particle}. In such instances, the following relationship can occur:

$$\frac{{( Fluidization Index )}_{catalyst particle}}{{( Fluidization Index )}_{inert particle}} < n$$

wherein “n” is defined as in equation (2).

In another embodiment, the (Fluidization Index)_{inert particle} can be <the (Fluidization Index)_{catalyst particle}. In such instances, the following relationship can occur:

$$\frac{{( Fluidization Index )}_{inert particle}}{{( Fluidization Index )}_{catalyst particle}} < n$$

wherein “n” is defined as in equation (2) above.

### D. Heating the Inert Material

The inert material provides at least a portion of the required heat for increasing sensible heat of the feedstock and/or converting at least a portion of the acyclic hydrocarbons to the first effluent comprising alkenes, cyclic hydrocarbons and/or aromatics, particularly converting acyclic C₅ hydrocarbons to cyclopentadiene. Thus, the process comprises removing an inert material-rich stream from the at least one reaction zone and heating the inert material-rich stream to produce a heated inert material-rich stream, which may be provided to the at least one reaction zone. As used herein, the term “inert material-rich stream” refers to a stream containing a greater amount of inert material than catalyst material.

Advantageously, due the differences in size and/or density between the inert material and the catalyst material, the inert material and the catalyst material may be substantially separated and isolated prior to heating. Thus, substantially only the inert material may be heated and reintroduced into the at least one reaction zone in order to avoid deactivating the catalyst material under oxidative heating conditions and/or minimize exposure to higher temperatures present in the re-heating vessel. In other words, the inert material-rich stream removed from the at least one reaction zone, which may be heated and provided back to the at least one reaction zone may be considered a heating loop, which is separate and different from a catalyst recirculation loop as further described below.

Therefore, following removal from the at least one reaction zone, catalyst material may optionally be separated from the inert material-rich stream by any suitable methods, for example, via standard stripping methods. Examples of suitable stripping gases that may be used include, but are not limited to hydrogen, light hydrocarbons (C₁-C₄) (e.g., methane), and combinations thereof. The catalyst material removed or stripped from the inert material-rich stream may then be provided back into the at least one reaction zone. Preferably, such catalyst material is provided to the at least one reaction zone at a position above where the inert material-rich stream is removed.

Once separated from the catalyst material, the inert material-rich stream may then be heated by any suitable means to produce the heated inert material. For example, the inert material-rich stream may be contacted with a flue gas, for example, in a reactor, such as a combustion riser. The flue gas may be generated by any suitable means known in the art. For example, the flue gas may be generated by direct injection of a fuel gas (e.g., methane) to achieve a desired adiabatic temperature rise and axially distributing air in the riser to gradually raise the temperature of the flue gas. Additionally or alternatively, the flue gas may be generated externally as a product of combustion from a furnace, gas turbine, or catalytic combustion and may optionally be diluted with a portion of the cooled flue gas (recirculated after heat recovery) or air to cool the flue gas temperature to desired temperature before contacting with inert solids. The hot flue gas may be contacted with the inert material by distributing the hot flue gas axially along the riser to gradually raise the temperature of the inert material.

Additionally, or alternatively, the inert material-rich stream may be contacted with pre-heated hydrogen and/or light hydrocarbons (C₁-C₄), for example, pre-heated in a furnace to a desirable temperature. In some embodiments, during heating of the inert material-rich stream, the pre-heated hydrogen and/or light hydrocarbons (C₁-C₄) cools to become a cooled hydrogen and/or light hydrocarbons (C₁-C₄) stream. This cooled hydrogen and/or light hydrocarbons stream may be used as the above-described stripping gas to remove the catalyst material from the inert material-rich stream. Optionally, a portion of the cooled hydrogen and/or light hydrocarbons stream may be compressed and recirculated through a furnace to be heated and re-used as the pre-heated hydrogen and/or light hydrocarbons (C₁-C₄) for heating the inert material-rich stream.

The inert material-rich stream may be contacted with the flue gas or the hydrogen and/or light hydrocarbon (C₁-C₄) at a temperature of ≥about 400° C., ≥about 500° C., ≥about 600° C., ≥about 700° C., ≥about 800° C., ≥about 900° C., ≥about 1000° C., ≥about 1100° C., ≥about 1200° C., about ≥1300° C., about ≥1400° C., about ≥1500° C., about ≥1600° C., about ≥1700° C., about ≥1800° C., about ≥1900° C., about ≥2000° C., about ≥2100° C., or about ≥2200° C. In particular, the inert material may be contacted with the flue gas or the hydrogen and/or light hydrocarbon (C₁-C₄) at a temperature of ≥about 600° C. Ranges of temperatures expressly disclosed include combinations of any of the above-enumerated values, e.g., about 400° C. to about 900° C., about 500° C. to about 800° C., about 600° C. to about 900° C., etc. Preferably, the inert material-rich stream may be contacted with the flue gas or the hydrogen and/or light hydrocarbon (C₁-C₄) at a temperature of about 400° C. to about 2200° C., more preferably about 500° C. to about 1500° C., or more preferably about 600° C. to about 1200° C.

In various aspects, the process may further comprise removing flue gas from the heated inert material by any suitable means prior to providing the heated inert material-rich stream to at least one reaction zone. Removing flue gas from the heated inert material-rich stream may be performed in order to avoid introduction of oxygen-containing molecules present in the flue gas (e.g., H₂O, COₓ, etc.) into the at least one reaction zone where such oxygen-containing molecules can deactivate the catalyst material. Thus, in some aspects, the heated inert material-rich stream may be separated from combustion gas and/or flue gas via one or more cyclones, filter, electrostatic precipitators, and/or other gas solid separation equipment. Additionally or alternatively, the heated inert material-rich stream may be stripped with hydrogen and/or light hydrocarbon (C₁-C₄) to remove combustion gas and/or flue gas. In some embodiments, where the inert material-rich stream is heated via contact with pre-heated hydrogen and/or light hydrocarbons (C₁-C₄), this further stripping of the resultant heated inert material may not be necessary.

In particular, the heated inert material-rich stream, which is provided to the at least one reaction zone, may provide ≥about 10%, ≥about 20%, ≥about 30%, ≥about 35%, ≥about 40% ≥about 45%, ≥about 50%, ≥about 55%, ≥about 60%, ≥about 65%, ≥about 70%, ≥about 75%, ≥about 80%, ≥about 85%, ≥about 90%, ≥about 95%, or 100% of the required heat for converting at least a portion of the acyclic hydrocarbons to the first effluent comprising alkenes, cyclic hydrocarbons and/or aromatics, particularly converting acyclic C₅ hydrocarbons to cyclopentadiene. In particular, the heated inert material-rich stream may provide ≥20% of the required heat for converting at least a portion of the acyclic C₅ hydrocarbons to the first effluent comprising cyclopentadiene. Ranges expressly disclosed include combinations of any of the above-enumerated values; e.g., about 20% to about 100%, about 40% to about 95%, about 50% to about 90%, etc. Preferably, the heated inert material-rich stream may provide about 20% to about 100% of the required heat, more preferably 40% to about 100% of the required heat, or more preferably 50% to about 100% of the required heat

### E. Effluent

An effluent (e.g., first effluent, second effluent) exiting the at least one reaction zone may comprise a variety of hydrocarbon compositions produced from the reaction of the acyclic hydrocarbons (e.g., acyclic C₅ hydrocarbons) in the at least one reaction zone. The hydrocarbon compositions typically have mixtures of hydrocarbon compounds, such as alkenes, cyclic hydrocarbons, and aromatics, having from 1 to 30 carbon atoms (C₁-C₃₀ hydrocarbons), from 1 to 24 carbon atoms (C₁-C₂₄ hydrocarbons), from 1 to 18 carbon atoms (C₁-C₁₈ hydrocarbons), from 1 to 10 carbon atoms (C₁-C₁₀ hydrocarbons), from 1 to 8 carbon atoms (C₁-C₅ hydrocarbons), and from 1 to 6 carbon atoms (C₁-C₆ hydrocarbons). Particularly, the first effluent comprises cyclopentadiene. The cyclopentadiene may be present in a hydrocarbon portion of an effluent (e.g., first effluent, second effluent) in an amount of ≥about 20.0 wt %, ≥about 25.0 wt %, ≥about 30.0 wt %, ≥about 35.0 wt %, ≥about 40.0 wt %, ≥about 45.0 wt %, ≥about 50.0 wt %, ≥about 55.0 wt %, ≥about 60.0 wt %, ≥about 65.0 wt %, ≥about 70.0 wt %, ≥about 75.0 wt %, or ≥about 80.0 wt %. Additionally or alternatively, the cyclopentadiene may be present in a hydrocarbon portion of an effluent (e.g., first effluent, second effluent) in an amount of ≤about 20.0 wt %, ≤about 25.0 wt %, ≤about 30.0 wt %, ≤about 35.0 wt %, ≤about 40.0 wt %, ≤about 45.0 wt %, ≤about 50.0 wt %, ≤about 55.0 wt %, ≤about 60.0 wt %, ≤about 65.0 wt %, ≤about 70.0 wt %, ≤about 75.0 wt %, ≤about 80.0 wt %, or ≤about 85.0 wt %. Ranges expressly disclosed include combinations of any of the above-enumerated values, e.g., about 20.0 wt % to about 85.0 wt %, about 30.0 wt % to about 75.0 wt %, about 40.0 wt % to about 85.0 wt %, about 50.0 wt % to about 85.0 wt %, etc. Preferably, the cyclopentadiene may be present in a hydrocarbon portion of an effluent (e.g., first effluent, second effluent) in an amount of about 10.0 wt % to about 85.0 wt %, more preferably about 25.0 wt % to about 80.0 wt %, more preferably about 40.0 wt % to about 75.0 wt %.

In other aspects, an effluent (e.g., first effluent, second effluent) may comprise one or more other C₅ hydrocarbons in addition to cyclopentadiene. Examples of other C₅ hydrocarbons include, but are not limited to cyclopentane and cyclopentene. The one or more other C₅ hydrocarbons may be present in a hydrocarbon portion of an effluent (e.g., first effluent, second effluent) in an amount ≥about 10.0 wt %, ≥about 15.0 wt %, ≥about 20.0 wt %, ≥about 25.0 wt %, ≥about 30.0 wt %, ≥about 35.0 wt %, ≥about 40.0 wt %, ≥about 45.0 wt %, ≥about 50.0 wt %, ≥about 55.0 wt %, ≥about 60.0 wt %, ≥about 65.0 wt %, or ≥about 70.0 wt %. Additionally or alternatively, the one or more other C₅ hydrocarbons may be present in a hydrocarbon portion of an effluent (e.g., first effluent, second effluent) in an amount of ≤about 15.0 wt %, ≤about 20.0 wt %, ≤about 25.0 wt %, ≤about 30.0 wt %, ≤about 35.0 wt %, ≤about 40.0 wt %, ≤about 45.0 wt %, ≤about 50.0 wt %, ≤about 55.0 wt %, ≤about 60.0 wt %, ≤about 65.0 wt %, or ≤about 70.0 wt %. Ranges expressly disclosed include combinations of any of the above-enumerated values, e.g., about 10.0 wt % to about 70.0 wt %, about 10.0 wt % to about 55.0 wt %, about 15.0 wt % to about 60.0 wt %, about 25.0 wt % to about 65.0 wt %, etc. Preferably, the one or more other C₅ hydrocarbons may be present in a hydrocarbon portion of an effluent (e.g., first effluent, second effluent) in an amount of about 30.0 wt % to about 65.0 wt %, more preferably about 20.0 wt % to about 40.0 wt %, more preferably about 10.0 wt % to about 25.0 wt %.

In other aspects, an effluent (e.g., first effluent, second effluent) may also comprise one or more aromatics, e.g., having 6 to 30 carbon atoms, particularly 6 to 18 carbon atoms. The one or more aromatics may be present in a hydrocarbon portion of an effluent (e.g., first effluent, second effluent) in an amount of about ≥about 1.0 wt %, ≥about 5.0 wt %, ≥about 10.0 wt %, ≥about 15.0 wt %, ≥about 20.0 wt %, ≥about 25.0 wt %, ≥about 30.0 wt %, ≥about 35.0 wt %, ≥about 40.0 wt %, ≥about 45.0 wt %, ≥about 50.0 wt %, ≥about 55.0 wt %, ≥about 60.0 wt %, or ≥about 65.0 wt %. Additionally, or alternatively, the one or more aromatics may be present in a hydrocarbon portion of an effluent (e.g., first effluent, second effluent) in an amount of ≤about 1.0 wt %, ≤about 5.0 wt %, ≤about 10.0 wt %, ≤about 15.0 wt %, ≤about 20.0 wt %, ≤about 25.0 wt %, ≤about 30.0 wt %, ≤about 35.0 wt %, ≤about 40.0 wt %, ≤about 45.0 wt %, ≤about 50.0 wt %, ≤about 55.0 wt %, ≤about 60.0 wt %, or ≤about 65.0 wt %. Ranges expressly disclosed include combinations of any of the above-enumerated values, e.g., about 1.0 wt % to about 65.0 wt %, about 10.0 wt % to about 50.0 wt %, about 15.0 wt % to about 60.0 wt %, about 25.0 wt % to about 40.0 wt %, etc. Preferably, the one or more aromatics may be present in a hydrocarbon portion of an effluent (e.g., first effluent, second effluent) in an amount of about 1.0 wt % to about 15.0 wt %, more preferably about 1.0 wt % to about 10 wt %, more preferably about 1.0 wt % to about 5.0 wt %.

For information on possible dispositions of the effluents, please see applications: U.S. Ser. No. 62/250,678, filed Nov. 4, 2015; U.S. Ser. No. 62/250,692, filed Nov. 4, 2015; U.S. Ser. No. 62/250,702, filed Nov. 4, 2015; and U.S. Ser. No. 62/250,708, filed Nov. 4, 2015; which are incorporated herein by reference.

### F. Stripping/Separation of the Effluent

In various aspects, catalyst material and/or inert material may become entrained with hydrocarbons (e.g., cyclopentadiene) in the effluent (e.g., first effluent, second effluent) as the effluent travels through and/or exits the at least one reaction zone. Thus, the process may further comprise separating catalyst material and/or inert material, which may be entrained with hydrocarbons (e.g., cyclopentadiene) in the effluent (e.g., first effluent, second effluent). This separating may comprise removal of the catalyst material and/or inert material from the hydrocarbons (e.g., cyclopentadiene) by any suitable means, such as, but not limited to cyclones, filter, electrostatic precipitators, heavy liquid contacting, and/or other gas solid separation equipment, which may be inside and/or outside the at least one reaction zone. The effluent free of particulate material may then travel to a product recovery system. Additionally, the separated catalyst material and/or inert material may then be fed back into the at least one reaction zone, for example, in a substantially top portion of the at least one reaction zone using known methods. This separation of catalyst material from the effluent and reintroduction back into the at least one reaction zone may be considered a catalyst recirculation loop. In a particular embodiment, a separated catalyst material stream may be introduced into the at least one reaction zone at a position above where the inert material-rich stream is removed from the at least one reaction zone, which may minimize catalyst material from exiting the at least one reaction zone along with the inert material.

Additionally, or alternatively, the separated material with reduced level of hydrocarbons may then travel to a rejuvenation zone, and/or regeneration zone, and the hydrocarbons stripped from the particulate material may be directed to the product recovery system or to the reactor system.

### G. Rejuvenation

As the reaction occurs in the at least one reaction zone, coke material may form on the particulate material, particularly on the catalyst material, which may reduce the activity of the catalyst material. Additionally or alternatively, the particulate material may cool as the reaction occurs. The catalyst material exiting the at least one reaction zone is referred to as “spent catalyst material.” Thus, the effluent and the separate catalyst material can comprise spent catalyst material. This spent catalyst material may not necessarily be a homogenous mix of particles as individual particles may have had a distribution of total aging in the system, time since last regeneration and/or rejuvenation, and/or ratio of times spent in reaction zones relative to in the regeneration and/or rejuvenation zones.

Thus, at least a portion of the particulate material (e.g., spent catalyst material) may be transferred from the at least one reaction zone to a rejuvenation zone to produce rejuvenated catalyst material. The transferring of the particulate material (e.g., spent catalyst material) from the at least one reaction zone to a reheating zone may occur after the catalyst material has been stripped and/or separated from the hydrocarbons after exiting the at least one reaction zone. Additionally or alternatively, catalyst (e.g., spent catalyst material) material may be transferred directly from the at least one reaction zone to a reheating zone. The reheating zone may include one more heating devices, such as but not limited to direct contacting, a heating coil, and/or a fired tube.

In various aspects, in the rejuvenation zone, the particulate material (e.g., spent catalyst material) may be contacted with a gaseous stream comprising hydrogen and substantially free of reactive oxygen-containing compounds to remove at least a portion of incrementally deposited coke material on the catalyst material thereby forming a rejuvenated catalyst material and a volatile hydrocarbon, such as, but not limited to methane. As used herein, the term “incrementally deposited” coke material refers to an amount of coke material that is deposited on the catalyst material during each pass of the catalyst material through the at least one reaction zone as opposed to a cumulative amount of coke material deposited on the catalyst material during multiple passes through the at least one reaction zone. “Substantially free” used in this context means the rejuvenation gas comprises less than about 1.0 wt %, based upon the weight of the gaseous stream, e.g., less than about 0.1 wt %, less than about 0.01 wt %, less than about 0.001 wt %, less than about 0.0001 wt %, less than about 0.00001 wt % oxygen-containing compounds. “Reactive oxygen-containing compounds” are compounds where the oxygen is available to react with the catalyst as compared to inert compounds containing oxygen (such as CO), which do not react with the catalyst. The gaseous stream may comprise ≥50 wt % H₂, such as ≥60 wt %, ≥70 wt %, preferably ≥90 wt % H₂. The gaseous stream may further comprise an inert substance (e.g., N₂, CO), and/or methane. Contacting the spent catalyst material with the gaseous stream may occur at a temperature of about 500° C. to about 900° C., preferably about 575° C. to about 750° C. and/or at a pressure between about 5.0 psia to about 250 psia, preferably about 25 psia to about 250 psia.

In alternative aspects, in the rejuvenation zone, the particulate material (e.g., spent catalyst material) may be rejuvenated via a mild oxidation procedure comprising contacting the particulate material with an oxygen-containing gaseous stream under conditions effective to remove at least a portion of incrementally deposited coke material on the catalyst material thereby forming a rejuvenated catalyst material. Typically, these conditions include a temperature range of about 250° C. to about 500° C., and a total pressure of about 0.1 bar to about 100 bar, preferably at atmospheric pressure. Further, the oxygen-containing gaseous stream is typically supplied to the rejuvenation zone at a total WHSV in the range of about 1 to 10,000. Following the mild oxidation, purge gas is generally reintroduced to purge oxidants from the catalyst composition using a purge gas, for example, N₂. This purging step may be omitted if CO₂ is the oxidant as it will no produce a flammable mixture. Optionally, rejuvenation via mild oxidation further comprises one or more hydrogen treatment steps.

In any embodiment, the rejuvenated catalyst material may then be returned to the at least one reaction zone.

In any embodiment, rejuvenation is generally effective at removing at least 10 wt % (≥10 wt %) of incrementally deposited coke material. Between about 10 wt % to about 100 wt %, preferably between about 60 wt % to about 100 wt %, more preferably between about 90 wt % to about 100 wt % of incrementally deposited coke material is removed.

Rejuvenation advantageously may have a time duration of ≤90 mins, e.g., ≤60 mins, ≤30 mins, ≤10 mins, such as ≤1 min, or ≤10 seconds. Rejuvenation may be advantageously performed ≥10 minutes, e.g., ≥30 minutes, ≥2 hours, ≥5 hours, ≥24 hours, ≥2 days, ≥5 days, ≥20 days, after beginning the specified conversion process.

Rejuvenation effluent exiting the rejuvenation zone and comprising, unreacted hydrogen, coke particulate, and optionally light hydrocarbon, may be further processed. For example, in aspects where rejuvenation is achieved via contact with a hydrogen-rich gaseous stream, the rejuvenation effluent may be sent to a compression device and then sent to a separation apparatus wherein a light hydrocarbon enriched gas and light hydrocarbon depleted gas is produced. The light hydrocarbon gas may be carried away, e.g., for use as fuel gas. The light hydrocarbon depleted stream may be combined with make-up hydrogen and make up at least a portion of the gaseous stream provided to the rejuvenation zone. The separation apparatus may be a membrane system, adsorption system (e.g., pressure swing or temperature swing), or other known system for separation of hydrogen from light hydrocarbons. A particulate separation device, e.g., a cyclonic separation drum, may be provided wherein coke particulate is separated from the rejuvenation effluent.

### H. Regeneration

The process may further comprise a regeneration step to recapture catalyst activity lost due to the accumulation of coke material and/or agglomeration of metal on the catalyst material during the reaction. This regeneration step may be carried out when there has not been sufficient removal of the coke material from the particulate material (e.g., spent catalyst material) in the rejuvenation zone.

Preferably, in the regeneration step, at least a portion of the spent catalyst material from the at least one reaction zone, from the separated catalyst material following stripping from the effluent, and/or from the rejuvenation zone may be transferred to a regeneration zone and regenerated by methods known in the art. For example, an oxidative regeneration may be used to remove at least a portion of coke material from the spent catalyst material. In various aspects, a regeneration gas comprising an oxidizing material such as oxygen, for example, air, may contact the spent catalyst material. The regeneration gas may oxidatively remove at least 10 wt % (≥10 wt %) of the total amount of coke material deposited on the catalyst composition at the start of regeneration. Typically, an oxychlorination step is performed following coke removal comprising contacting the catalyst composition with a gaseous stream comprising a chlorine source and an oxygen source under conditions effective for dispersing at least a portion of metal, e.g., Group 10 metal, particles on the surface of the catalyst and to produce a metal chlorohydrate, e.g., a Group 10 metal chlorohydrate. Additionally, a chlorine stripping step is typically performed following oxychlorination comprising contacting the catalyst composition with a gaseous stream comprising an oxygen source, and optionally a chlorine source, under conditions effective for increasing the O/Cl ratio of the metal chlorohydrate. Generally, a reduction step, and optionally a sulfidation step may also be performed in the regeneration step. Typically, regeneration is effective at removing between about 10 wt % to about 100 wt %, preferably between about 90 wt % to about 100 wt % of coke material is removed. Optionally, before or after contacting the spent catalyst material with the regeneration gas, the catalyst material may also be contacted with a purge gas, e.g., N₂. Regeneration, including purging before and after coke oxidation, requires less than 10 days, preferably less than about 3 days to complete.

Catalyst may be continuously withdrawn from and returned to the reaction zone and/or the rejuvenation zone or may be periodically withdrawn from and returned to the reaction zone and/or regeneration zone. For a periodic method, typically, the regeneration times between when withdrawals are made for coke burn, oxychlorination, chlorine stripping, purge, reduction, and optional sulfidation occurs are between about 24 hours (about 1 day) to about 240 hours (about 10 days), preferably between about 36 hours (about 1.5 days) to about 120 hours (about 5 days). Alternatively for continuous mode, the removal/addition of particulate material rate may vary between about 0.0 wt % to about 100 wt % (e.g., about 0.01 wt % to about 100 wt %) per day of the particulate material inventory, and preferably between about 0.25 wt % to about 30.0 wt % per day of the particulate material inventory where there is balanced addition/removal of particulate material. Regeneration of the catalyst material may occur as a continuous process or may be done batch wise in both cases intermediate vessels for inventory accumulation and/or inventory discharge may be required.

The removal and addition of the particulate material (e.g., spent catalyst material, fresh catalyst material, fresh inert material, rejuvenated catalyst material, regenerated catalyst material) may occur at the same or different location in the reactor system. The particulate material (e.g., fresh catalyst material, fresh inert material, rejuvenated catalyst material, regenerated catalyst material) may be added after or before the rejuvenation zone, while the removal of the particulate material (e.g., spent catalyst material) may be done before or after the particulate material (e.g., spent catalyst material) is passed through the rejuvenation zone. At least a portion of the regenerated catalyst material may be recycled to the at least one reaction zone or at least one rejuvenation zone. Preferably, the regenerated catalyst material and/or fresh particulate material are provided to the rejuvenation zone to minimize any loss in heat input and to remove any remaining species that may be carried by the regenerated catalyst material from the regeneration zone. Additionally, or alternatively, separators inside or outside of the regeneration zone may be used to separate the inert material from the catalyst material prior to regeneration so that just the catalyst material is regenerated. This separation may be carried out on the basis of size, magnetic, and/or density property differences between the inert material and the regenerated catalyst material using any suitable means.

For the above-described processes, standpipes, well known by those skilled in the art with the particle size and operating conditions described above, may be used to provide the means of transporting the particulate material between the at least one reaction zone, rejuvenation zone, and/or regeneration zone. Slide valves and lifting gas, known by those skilled in the art, may also be used to help circulate the particulate material and/or build the necessary pressure profile inside the regeneration zone. The lifting gas may be the same as the fluidizing gas used in the rejuvenation zone, e.g., a hydrogen stream that may contribute in minimizing the hydrogen usage in the reaction system, while also reducing the coke material formation.

### III. REACTION SYSTEMS FOR CONVERSION OF ACYCLIC HYDROCARBONS

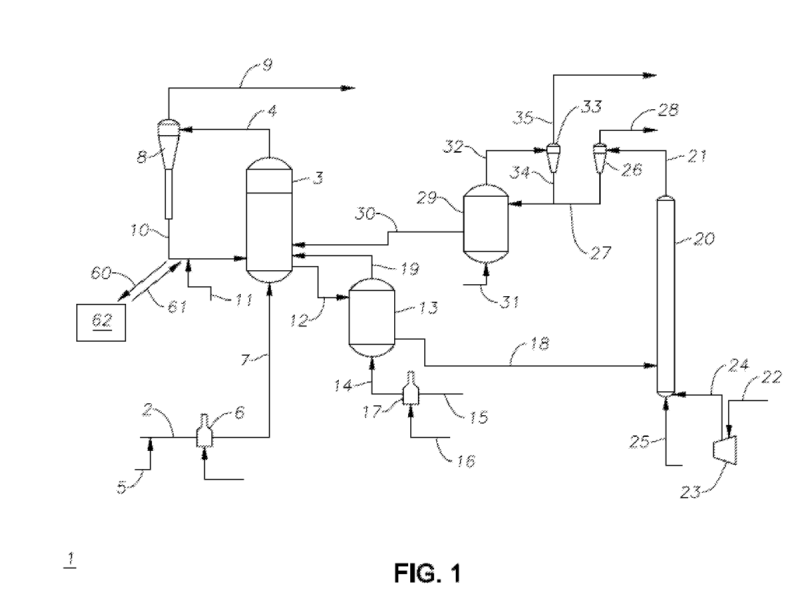

In another embodiment, a reaction system 1 for converting acyclic hydrocarbons (e.g., acyclic C₅ hydrocarbons) to alkenes, cyclic hydrocarbons (e.g., cyclopentadiene) and/or aromatics is provided, as shown in FIG. 1. The reaction system 1 may comprise a feedstock stream 2 comprising acyclic hydrocarbons (e.g., acyclic C₅ hydrocarbons, such as pentane) as described above, at least one reactor 3 as described above comprising catalyst material and inert material as described above, and an effluent stream 4 comprising alkenes, cyclic hydrocarbons (e.g., cyclopentadiene), and aromatics. In particular, the catalyst material and the inert material have a different average diameter and/or density as described above such that the catalyst material and the inert material exhibit different fluidization behavior as described above. The at least one reactor 3 may comprise a feedstock stream inlet (not shown) for providing the feedstock stream 2 to the reaction system and an effluent stream outlet (not shown) for removal of the effluent stream 4. Optionally, the reaction system 1 may further comprise a first furnace 6 for heating the feedstock stream 2 to produce a heated feedstock stream 7, which may be provided to the at least one reactor 3 at feedstock temperatures as described herein (e.g., ≤about 650° C.). Optionally, a first hydrogen stream 5 may be co-fed with the feedstock stream 2.

The at least one reactor 3 may be a circulating fluidized bed reactor. Additionally, or alternatively, the at least one reactor is not a radial-flow reactor or a cross-flow reactor.

Additionally, or alternatively, the at least one reactor 3 may comprise at least a first reactor, a second reactor, a third reactor, a fourth reactor, a fifth reactor, a sixth reactor, a seventh reactor, etc. As used herein, each “reactor” may be individual vessels or individual reaction zones within a single vessel. Preferably, the reaction system includes 1 to 20 reactors, more preferably 1 to 15 reactors, more preferably 2 to 10 reactors, more preferably 3 to 8 reactors. A circulating fluidized bed reactor may include multiple reaction zones (e.g., 3-8) within a single vessel or multiple vessels (e.g., 3-8). Where the reaction system includes more than one reactor, the reactors may be arranged in any suitable configuration, preferably in series, wherein a bulk of the feedstock moves from the first reactor to the second reactor and/or a bulk of the particulate material moves from the second reactor to the first reactor, and so on. Each reactor, independently, may be a circulating fluidized bed reactor or a circulating settling bed reactor.

Preferably, the at least one reactor 3 may include at least one or more internal structures 8, as described above. Particularly, the at least one reactor 3 may include a plurality of internal structures (e.g., 2, 3, 4, 5, 6, 7, 8, 9, 10, 15, 20, 30, 40, 50, etc.), such as, baffles, sheds, trays, tubes, tube bundles, tube coils, rods, and/or distributors.

The at least one reactor 3 is operated under reaction conditions as described above to convert at least a portion of the acyclic hydrocarbons (e.g., acyclic C₅ hydrocarbons) to alkenes, cyclic hydrocarbons (e.g., cyclopentadiene), and/or aromatics. For example, the reaction conditions may comprise a temperature of about 400° C. to about 700° C. and/or a pressure of about 3.0 psia to about 100 psia. Preferably, at least about 30 wt % of the acyclic C₅ hydrocarbons is converted to cyclopentadiene. Optionally, the at least one reactor 3 may include one or more heating devices (e.g., fired tube, heated coil) (not shown) in order to maintain temperature therein.

Additionally, the reaction system 1 may further comprise a first separator 8, such as a cyclone, (one is shown, but two or more operating in series may be present with one or more operating in parallel) in fluid connection with the at least one reactor 3. The first separator 8, may separate the catalyst material, which may be entrained with hydrocarbons (e.g., cyclopentadiene) in the effluent stream 4 to produce a separated catalyst material stream 10 and a catalyst-free effluent stream 9, substantially free of catalyst material. The catalyst-free effluent stream 9 may optionally travel to a product recovery system. Additionally, the separated catalyst material stream 10 may then be fed back into the at least one reactor 3 (the material may be returned to the top of the reactor but, more preferably, may be returned closer to the bottom of the reactor but located above the inert stream effluent 12 (described below)) via a separated catalyst material inlet (not shown) in the at least one reactor 3. The first separator 8 may comprise an effluent stream inlet (not shown), a separated catalyst material stream outlet (not shown), and a catalyst-free effluent stream outlet (not shown). Optionally, a second hydrogen stream 11 may be present in the reaction system 1, which may be fed to the first separator 8 and/or combined with the separated catalyst material stream 10.

Additionally, the reaction system 1 may further comprise an inert material-rich stream 12, which may exit the at least one reactor 3 via an inert material-rich stream outlet (not shown) and be heated and reintroduced into the at least one reactor 3. In various aspects, the separated catalyst stream inlet is at a position in the at least one reactor 3 above the inert material-rich stream outlet.

The reaction system 1 may comprise a second separator 13, such as a stripping vessel, for separating catalyst material from the inert material-rich stream 12, wherein the second separator 13 is in fluid connection with the at least one reactor 3. In various aspects, a first stripping gas stream 14 may be provided to the second separator 13 via a first stripping gas inlet (not shown). Any suitable stripping gas as described herein may be used as the stripping gas stream 14. For example, a hydrogen stream 16 and methane stream 15 may optionally be heated in a second furnace 17 to produce the stripping gas stream 14. A stripped inert material-rich stream 18 and a stripped catalyst material stream 19 may exit the second separator 13 via a stripped inert material-rich stream outlet (not shown) and a stripped catalyst material stream outlet (not shown). The stripped catalyst material stream 19 may be provided to the at least one reactor 3.

In order to produce a heated inert material-rich stream 21, the reaction system 1 may further comprise a second reactor 20, such as a combustion riser, in fluid connection with the second separator 13 and the at least one reactor 3 where the stripped inert material-rich stream 18 may be provided to the second reactor 20 via a stripped inert material-rich stream inlet (not shown). In the second reactor 20, the inert material may be contacted with a flue gas as described herein (e.g., at a temperature of ≥about 600° C.). For example, an air stream 22 may be compressed in a first compressor 23, and a compressed air stream 24 and a fuel gas stream 25 may be injected directly into the second reactor 20 to produce the flue gas which may contact and heat the stripped inert material to produce the heated inert material-rich stream 21 (e.g., having a temperature of ≥about 550° C.).

Optionally, the reaction system 1 may further comprise a fourth separator for removing flue gas from the heated inert material-rich stream 21. For example, the fourth separator may comprise a first cyclone 26 and a stripping vessel 29. The heated inert material-rich stream 21 may be provided to the first cyclone 26 to produce a separated heated inert material-rich stream 27 and a first separated flue gas stream 28. Additional flue gas may be removed from the separated heated inert material-rich stream 27 in the stripping vessel 29 to produce a stripped heated inert material-rich stream 30 and a stripping vessel effluent 32. A second stripping gas stream 31 as described herein may be provided to the stripping vessel 29. Any entrained inert material particles in the stripping vessel effluent 32 may be separated in a second cyclone 33 whereby a second separated flue gas stream 35, optionally comprising stripping gas, and inert material particles 34 are obtained. The inert material particles 34 may be reintroduced to the stripping vessel 29, optionally in combination with the separated heated inert material-rich stream 27. Following further separation, the stripped heated inert material-rich stream 30 may be introduced into the at least one reactor 3 via heated inert material-rich stream inlet (not shown).

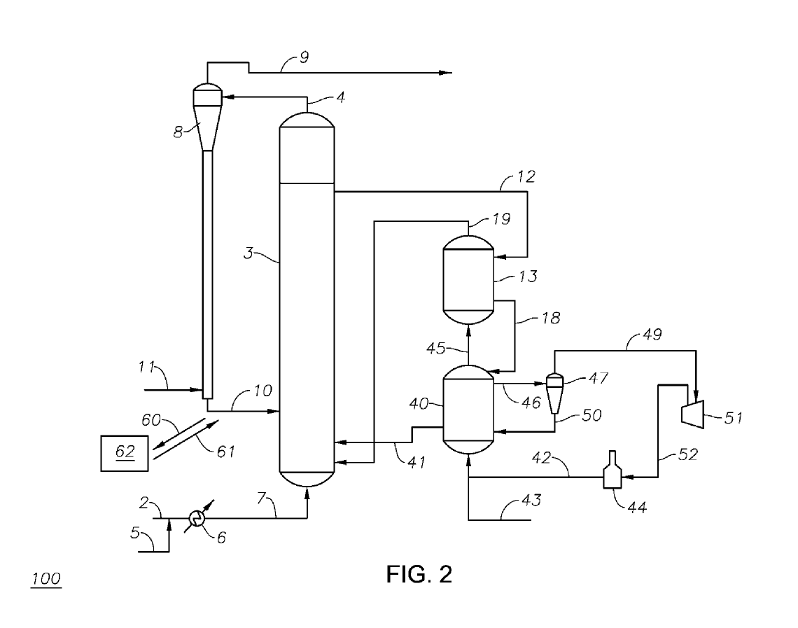

In an alternative embodiment, as shown in FIG. 2, instead of heating the stripped inert material-rich stream 18 with a flue gas in a reactor, a reaction system 100 is provided comprising a heating vessel 40, in fluid connection with the second separator 13 and the at least one reactor 3, to produce a heated inert material-rich stream 41. Thus, the reaction system 100 may comprise a first pre-heated gas stream 42, optionally heated via a second furnace 44, and optionally a co-fed second gas stream 43, which may be provided to the heating vessel 40 via a pre-heated gas stream inlet (not shown). The first pre-heated gas stream 42 and the co-fed second gas stream 43 may comprise hydrogen and/or light hydrocarbons (C₁-C₄) (e.g., methane) as described herein at a temperature as described herein (e.g., ≥about 600° C.), which contact the stripped inert material as it travels through the heating vessel 40 thereby exiting the vessel, preferably toward the bottom, as the heated inert material-rich stream 41, via a heated inert material-rich stream outlet (not shown). Advantageously, the heated inert material-rich stream 41 may not require further stripping or separation prior to introduction into the at least one reactor 3. In various aspects, the stripped heated inert material-rich stream 30 (FIG. 1) and/or the heated inert material-rich stream 41 (FIG. 2) may provide at least about 20% of required heat for converting at least a portion of the acyclic C₅ hydrocarbons to cyclopentadiene.

As the first pre-heated gas stream 42 and/or the co-fed second gas stream 43 travel through the heating vessel 40, they may cool and at least a portion of the first pre-heated gas stream 42 and/or the co-fed second gas stream 43 may exit the heating vessel 40 as a first cooled gas stream 45 comprising hydrogen and/or light hydrocarbons (C₁-C₄) via a first cooled gas stream outlet (not shown). This first cooled gas stream 45 may be provided to the second separator 13 for use as a stripping gas. Optionally, at least another portion of the first pre-heated gas stream 42 and/or the co-fed second gas stream 43 may exit the heating vessel 40 as a second cooled gas stream 46 comprising hydrogen and/or light hydrocarbons (C₁-C₄) via a second cooled gas stream outlet (not shown). A third cyclone 47 may be present to remove entrained inert particles from the second cooled gas stream 46. The separated inert particles 50 may be provided back to the heating vessel 40 and the separated second cooled gas stream 49 may be recycled for use as the first pre-heated gas stream 42. For example, a second compressor 51 may be present in the reaction system 100 for producing a compressed gas stream 52 that is heated in the second furnace 44 to produce the first pre-heated gas stream 42.

Additionally, or alternatively, the reaction system 1 and/or 100 may further comprise a rejuvenating and/or regenerating apparatus 62 for restoring activity of the spent catalyst material, wherein the rejuvenating and/or regenerating apparatus 62 is in fluid connection with the at least one reactor 3. For example, a spent catalyst stream 60 comprising at least a portion of the separated catalyst material stream 10 may be provided to the rejuvenating and/or regenerating apparatus 62 to produce a rejuvenated and/or regenerated catalyst stream 61.

Additionally, or alternatively, the reaction system 1 and/or 100 may further comprise a fresh particulate material stream (not shown) in fluid connection with the at least one reactor 3.

### IV. INDUSTRIAL APPLICABILITY

A first hydrocarbon reactor effluent obtained during the acyclic C₅ conversion process containing cyclic, branched and linear C₅ hydrocarbons and, optionally, containing any combination of hydrogen, C₄ and lighter byproducts, or C₆ and heavier byproducts is a valuable product in and of itself. Preferably, CPD and/or DCPD may be separated from the reactor effluent to obtain purified product streams which are useful in the production of a variety of high value products.

For example, a purified product stream containing 50 wt % or greater, or preferably 60 wt % or greater of DCPD is useful for producing hydrocarbon resins, unsaturated polyester resins, and epoxy materials. A purified product stream containing 80 wt % or greater, or preferably 90 wt % or greater of CPD is useful for producing Diels-Alder reaction products formed in accordance with the following reaction Scheme (I):

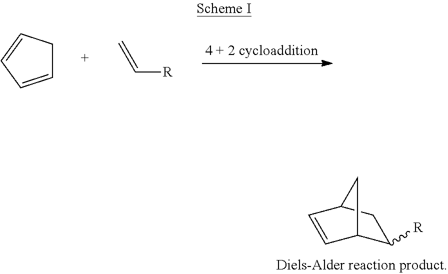

where R is a heteroatom or substituted heteroatom, substituted or unsubstituted C₁-C₅₀ hydrocarbyl radical (often a hydrocarbyl radical containing double bonds), an aromatic radical, or any combination thereof. Preferably, substituted radicals or groups contain one or more elements from Groups 13-17, preferably from Groups 15 or 16, more preferably nitrogen, oxygen, or sulfur. In addition to the mono-olefin Diels-Alder reaction product depicted in Scheme (I), a purified product stream containing 80 wt % or greater, or preferably 90 wt % or greater of CPD can be used to form Diels-Alder reaction products of CPD with one or more of the following: another CPD molecule, conjugated dienes, acetylenes, allenes, disubstituted olefins, trisubstituted olefins, cyclic olefins, and substituted versions of the foregoing. Preferred Diels-Alder reaction products include norbornene, ethylidene norbornene, substituted norbornenes (including oxygen-containing norbornenes), norbornadienes, and tetracyclododecene, as illustrated in the following structures:

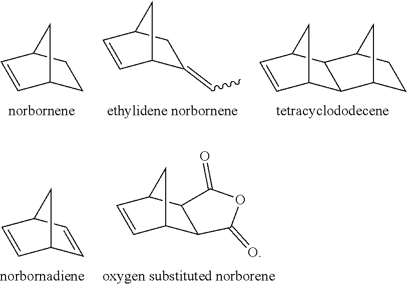

The foregoing Diels-Alder reaction products are useful for producing polymers and copolymers of cyclic olefins copolymerized with olefins such as ethylene. The resulting cyclic olefin copolymer and cyclic olefin polymer products are useful in a variety of applications, e.g., packaging film.

A purified product stream containing 99 wt % or greater of DCPD is useful for producing DCPD polymers using, for example, ring opening metathesis polymerization (ROMP) catalysts. The DCPD polymer products are useful in forming articles, particularly molded parts, e.g., wind turbine blades and automobile parts.

Additional components may also be separated from the reactor effluent and used in the formation of high value products. For example, separated cyclopentene is useful for producing polycyclopentene, also known as polypentenamer, as depicted in Scheme (II).

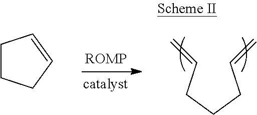

Separated cyclopentane is useful as a blowing agent and as a solvent. Linear and branched C₅ products are useful for conversion to higher olefins and alcohols. Cyclic and non-cyclic C₅ products, optionally after hydrogenation, are useful as octane enhancers and transportation fuel blend components.

All documents described herein are incorporated by reference herein, including any priority documents and/or testing procedures to the extent they are not inconsistent with this text. As is apparent from the foregoing general description and the specific embodiments, while forms of the invention have been illustrated and described, various modifications can be made without departing from the spirit and scope of the invention. Accordingly, it is not intended that the invention be limited thereby. Likewise, the term “comprising” is considered synonymous with the term “including.” Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

### PROPHETIC EXAMPLE

The following examples are derived from modeling techniques and although the work was actually achieved, the inventors do not present these examples in the past tense to comply with M.P.E.P. § 608.01(p) if so required.

### Example 1—Design of Reactor with Bimodal Catalyst Particles and Inert Particles

Reactor modeling in Example 1 was performed using Invensys Systems Inc. PRO/II 9.3.4 for the purpose of estimating reactor feed and product stream conditions and properties. Depending on specifics of the modeling, variation in results will occur but the models will still demonstrate the relative benefits of the present invention. Numerous modifications and variations are possible and it is to be understood that within the scope of the claims, the invention may be practiced otherwise than as specifically described herein.

The objective of Example 1 is to determine the relative difference in particle size and/or density necessary to impart different fluidization behavior for SiC inert particles and catalyst particles (e.g., spray-dried, formulated zeolite catalyst) in a fluidized bed reactor for producing cyclopentadiene (CPD) from pentane. Specifically, it is intended for the SiC inert particles to be in a bubbling or turbulent fluidization regime, while catalyst particles are intended to be in a dilute-phase, transport regime. All calculations for catalyst particles given below are performed based on a typical formulated zeolite based catalyst, e.g., a fluid catalytic cracking catalyst.

For a given SiC average particle size, the reactor diameter is sized to achieve the required gas velocity for turbulent fluidization. Once the reactor diameter is chosen, thereby fixing the superficial gas velocity, the catalyst particle size is selected to be below the maximum average particle size permissible to maintain the transport fluidization regime. The inert SiC particle has a density of 3400 kg/m³ and the zeolite catalyst particle has a density of 1400 kg/m³. Further parameters included are production of 200 kTA of CPD from a pentane feed in a fluidized bed reactor using the SiC inert particles and zeolite catalyst particles.

As shown below in Table 1, for SiC particles having a size of 100 μm, the reactor diameter is ˜14 feet, which results in a superficial gas velocity of 5.6 m/s. At this superficial gas velocity, a catalyst average particle size of 50 μm would be required for the transport regime, which requires a minimum transport velocity of 3.9 m/s, as shown in Table 2. It should be noted that given the large difference in particle density between the zeolite catalyst particles and the SiC inert particles, the required difference in particle size (100 μm v. 50 μm) is relatively small in order to achieve the difference in fluidization behavior.

The ratio of the fluidization index (as determined according to equations (1) and (2)) of the zeolite catalyst and SiC inert particles is calculated to be 0.10, as shown below:

$$\frac{{( Fluidization Index )}_{catalyst}}{{( Fluidization Index )}_{SiC inerts}} = \frac{1400 * {50}^{2}}{3400 * {100}^{2}} = 0.10$$

This calculated fluidization index ratio of 0.10 is significantly below the preferred ratio of 1.0, and also below the more preferred ratio 0.5, required to achieve different fluidization regimes.

TABLE 1
Fluidization Calculations for 100 μm SiC Particles

| SiC Particle Size | 100 | μm |
|---|---|---|
| Number of Trains | 1 | |
| Total Mass Rate | 14.5 | kg/s |
| Temperature | 575 | ° C. |
| Vapor Mas Rate | 14.5 | kg/s |
| Vapor Act. Density | 0.181 | kg/m³ |
| Vapor Viscosity | 2.31E−05 | kg/m · s |
| Vapor Act. Vol. Rate | 2.44E+08 | ft³/day |
| System Length Parameter (Diameter) | 4.04−E05 | m |
| System Rate Parameter (w) | 3.15 | m/s |
| Zenz Size Number (Zes) | 2.5 | dp/D |
| Geldart Particle Classification | A | |
| Archimedes Number (Ar) | 1.13E+01 | |
| Min. Fluidization Velocity (PSRI) | 0.009 | m/s |
| Min. Turbulent Regime (Uk) (Tsukada 1995) | 5.0 | m/s |
| Min. Churning Regime (Uc) (Horio 1986) | 3.8 | m/s |
| Min. Churning Regime (Uc) (Lee & Kim 1988) | 2.9 | m/s |
| Min. Churning Regime (Uc) (Bi & Grace 1995) | 4.7 | m/s |
| Gas Velocity Range, Min | 4.1 | m/s |
| Min. Transport Regime (Use) (Bi 1995) | 6.6 | m/s |
| Min. Transport Regime (Utr) (Tsukada 1995) | 7.0 | m/s |
| Min. Transport Regime (Utr) (Adenez 1993) | 8.1 | m/s |
| Gas Velocity Range, Max | 7.2 | m/s |
| Design Velocity Range, Avg | 5.7 | m/s |
| Reactor Diameter, D2 | 13.9 | ft |
| Reactor Diameter Setting | 14.0 | ft |
| Velocity if Diameter at Setting | 5.59 | m/s |

TABLE 2
Fluidization Calculations for 50 μm Zeolite Catalyst Particles

| Zeolite Particle Size | 50 | μm |
|---|---|---|
| Number of Trains | 1 | |
| Total Mass Rate | 14.5 | kg/s |
| Temperature | 575 | ° C. |
| Vapor Mass Rate | 14.5 | kg/s |
| Vapor Act. Density | 0.181 | kg/m³ |
| Vapor Viscosity | 2.31E−05 | kg/m · s |
| Vapor Act. Vol. Rate | 2.44E+08 | ft³/day |
| System Length Parameter (Diameter) | 5.44E−05 | m |
| System Rate Parameter (w) | 2.34 | m/s |
| Zenz Size Number (Zes) | 0.9 | dp/D |
| Geldart Particle Classification | AC | |
| Archimedes Number (Ar) | 5.83E−01 | |
| Min. Fluidization Velocity (PSRI) | 0.001 | m/s |
| Min. Transport Regime (Utr) (Tsukada 1995) | 3.6 | m/s |
| Min. Transport Regime (Utr) (Adenez 1993) | 4.1 | m/s |
| Min Gas Velocity for Transport Regime | 3.9 | m/s |

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the invention have been illustrated and described, various modifications can be made without departing from the spirit and scope of the invention. Accordingly, it is not intended that the invention be limited thereby. For example, the compositions described herein may be free of any component, or composition not expressly recited or disclosed herein. Any method may lack any step not recited or disclosed herein. Likewise, the term “comprising” is considered synonymous with the term “including.” And whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

1. A process for converting acyclic C₅ hydrocarbons to cyclopentadiene in a reactor system, wherein the process comprises:
contacting a feedstock comprising acyclic C₅ hydrocarbons with a catalyst material and an inert material in at least one reaction zone under reaction conditions to convert at least a portion of the acyclic C₅ hydrocarbons to a first effluent comprising cyclopentadiene, wherein the catalyst material and the inert material have a different average diameter and/or density, wherein the catalyst material is a crystalline aluminosilicate in combination with a Group 10 metal, Group 1 alkali metal and/or a Group 2 alkaline earth metal;
removing an inert material-rich stream comprising at least a first portion of the catalyst material from the at least one reaction zone;
optionally, separating at least a second portion of the catalyst material from the inert material-rich stream;
heating the inert material-rich stream to produce a heated inert material-rich stream; and
providing the heated inert material-rich stream to the at least one reaction zone.

2. The process of claim 1, wherein the at least one reaction zone is a circulating fluidized bed reactor.

3. The process of claim 1, wherein the feedstock is provided at a temperature of less than about 650° C. and/or the first effluent exiting the at least one reaction zone has a temperature of at least about 550° C.

4. The process of claim 1, wherein the heated inert material-rich stream has a temperature of at least about 550° C.

5. The process of claim 1, further comprising co-feeding hydrogen to the at least one reaction zone.

6. The process of claim 1, wherein the reaction conditions comprise a temperature of about 400° C. to about 700° C. and a pressure of about 3.0 psia to about 100 psia.

7. The process of claim 1, wherein the catalyst material and the inert material have the following relationship:
$$\frac{{( Fluidization Index )}_{particle 1}}{{( Fluidization Index )}_{particle 2}} < n$$
wherein particle 1 and particle 2 are the catalyst material particle or the inert material particle, provided that particle 1 and particle 2 are not the same and the (Fluidization Index)_{particle 1} is <(Fluidization Index)_{particle 2}; and n is 1.

8. The process of claim 7, wherein particle 1 is the catalyst material particle and particle 2 is the inert material particle.

9. The process of claim 1, wherein the catalyst material comprises platinum on ZSM-5, platinum on zeolite L, and/or platinum on silica.

10. The process of claim 1, wherein the catalyst material further comprises a binder comprising one or more of silica, titania, zirconia, metal silicates of Group 1 or Group 13 of the Periodic Table, carbides, nitrides, aluminum phosphate, aluminum molybdate, aluminate, surface passivated alumina, and mixtures thereof.

11. The process of claim 1, wherein the inert material comprises metal carbides, metal oxides, clays, metal phosphates, and a combination thereof.

12. The process of claim 1, wherein heating the inert material-rich stream comprises contacting the inert material-rich stream with:
(i) a flue gas at a temperature of at least about 600° C.; or
(ii) hydrogen and/or a C₁-C₄ hydrocarbon at a temperature of at least about 600° C.

13. (canceled)

14. The process of claim 13, wherein the separated catalyst material stream is introduced into the at least one reaction zone at a position above where the inert material-rich stream is removed from the at least one reaction zone.

15. The process of claim 1, further comprising transferring at least a portion of spent catalyst material to a rejuvenation zone and/or a regeneration zone to produce a rejuvenated catalyst material and/or a regenerated catalyst material; and returning the rejuvenated catalyst material and/or the regenerated catalyst material to the at least one reaction zone.

16. The process of claim 1, further comprising providing fresh inert material and/or fresh catalyst material to the at least one reaction zone.

17. The process of claim 1, further comprising removing flue gas from the heated inert material-rich stream prior to providing the heated inert material-rich stream to the at least one reaction zone.

18. (canceled)

19. The process of claim 18, wherein at least about 30 wt % of the acyclic C₅ hydrocarbons is converted to cyclopentadiene.

20. The process of claim 18, wherein the heated inert material-rich stream provides at least about 20% of required heat for converting at least a portion of the acyclic C₅ hydrocarbons to the first effluent comprising cyclopentadiene.

21.-25. (canceled)
